(19) United States
(12) Patent Application Publication
(10) Pub. No.: US 2025/0057484 A1
(43) Pub. Date: Feb. 20, 2025

(54) Objective and Training-Free Detection of High Frequency Oscillations in The Epileptic Brain

(71) Applicant: University of Kentucky Research Foundation, Lexington, KY (US)

(72) Inventors: Sridhar Sunderam, Lexington, KY (US); Amir F. Al-Bakri, Lexington, KY (US)

(21) Appl. No.: 18/235,142

(22) Filed: Aug. 17, 2023

Related U.S. Application Data
(60) Provisional application No. 63/398,616, filed on Aug. 17, 2022.

(51) Int. Cl. A61B 5/00; A61B 5/372

(52) U.S. Cl. CPC A61B 5/7264; A61B 5/372; A61B 5/4094; A61B 5/4836; A61B 5/4848

(57) ABSTRACT

A method of identifying high frequency oscillations (HFOs) in neural signal data involves detrending the neural signal data, and identifying HFOs through one or more objective and training-free strike tests selected from the group consisting of (i) amplitude, (ii) rhythmicity, and (iii) ringing.

### RELATED APPLICATIONS

This application claims priority from U.S. Provisional Application Ser. No. 63/398,616 filed Aug. 17, 2022, the entire disclosure of which is incorporated herein by this reference.

### GOVERNMENT INTEREST

This invention was made with government support under grant number 1539068 awarded by the National Science Foundation (NSF). The government has certain rights in the invention.

### TECHNICAL FIELD

The present disclosure is directed to methods for identifying high frequency oscillations. In particular, the disclosure is directed to a three strike algorithm for identifying high frequency oscillations with high specificity.

### Introduction

Many epilepsy patients suffer from seizures that are resistant to medication. Surgical resection may be a feasible option for some [2-4] but localization of the seizure onset zone (SOZ) from scalp electroencephalogram (EEG) or intracranial EEG (iEEG) recordings can be a tedious process: seizures, which are largely unpredictable events, need to be observed and analyzed in a clinical setting over several days.

High frequency oscillations (HFOs) are tiny neural discharges that are observed in electroencephalographic (EEG) recordings from the brains of epilepsy patients. Recent studies show that detecting and mapping HFOs can improve the prediction of which specific region (or regions) of the brain is likely to generate seizures and is therefore a candidate for surgical removal, particularly in patients who cannot be treated with drugs or alternative therapies. As such, HFOs are being investigated as biomarkers of epileptogenic brain tissue.

One attraction to the use of HFOs is that these brief electrophysiological events—commonly categorized as ripples (80-250 Hz) or fast ripples (250-500 Hz)—could be acquired from a brief (10-20 min) interictal iEEG recording and their activity used to predict the region of interest [5-7]. In a meta-analysis of relevant literature, patients with resected areas that were associated with high HFO density (either ripples or fast ripples) experienced better post-surgical outcomes than patients in whom the resected and HFO-active areas were discordant [8]. Whether HFOs are more useful as predictors of surgical outcome than seizures or interictal spikes is an open question, but it is hard to deny that they could contribute useful information to the diagnostic process at no additional expense.

The trouble with HFOs is in their appearance. They are brief (typically under 100 ms), of low amplitude relative to baseline activity, and high frequency by definition, which means that slower activity must be filtered out before they become evident. HFOs often occur in conjunction with interictal or ictal spikes [9-11]. Spikes themselves are relatively brief and sharp, and can produce false ripple-like oscillations when passed through high-frequency linear filters, an effect known as ringing [12]. Special care is required to avoid confusing ringing artifacts with true HFO activity and vice versa.

Expert visual inspection is regarded as the gold standard method of HFO verification [13-15]. However, the likelihood of encountering HFO activity in a given channel is minuscule [16]. A common rule of thumb is to analyze a 10-minute segment of iEEG during N3 sleep (i.e., stage 3 of non-rapid eye movement, or NREM, sleep), in which HFOs are more prevalent, to map HFO activity [17, 18]. But visual review of 10 min of iEEG in search of HFOs is labor-intensive and can take an hour to complete [15]. Some level of automation, if only to prescreen potential segments of interest for further review, is therefore desirable [19].

Several methods have been proposed to automate HFO analysis and differentiate HFOs from spikes and filtering artifacts with varying degrees of accuracy. Some require supervision (supervised approaches), which means that they are modeled and optimized on limited, labeled training data [20-23]. In other words, supervised approaches require labeled sample data and fit models to the data that seek differences between positive and negative samples. Some supervised approaches make use of heuristic approaches, which encode arbitrary criteria based on expert experience, implying foreknowledge of the properties and their typical values of HFOs. Heuristic parameters and criteria based on expert knowledge or intuition that have been tweaked or tuned to perform in the desired manner—and may therefore lack generality [1, 16, 24-27].

Other, unsupervised, modeling approaches look for natural partitions or clusters in a recording that differentiate HFOs from other activity assuming that HFOs are present in the sample [28, 29]. Unsupervised approaches find natural partitions in sample data, or the data to be analyzed, that may differentiate HFOs from non-HFOs, but with the assumption that the data contains both types of events.

Another limitation of some methods is that they are computationally intensive, which may slow down analysis. Given the variety of approaches, much work remains to be done before there is a consensus on the defining features of a quality HFO sample.

Accordingly, there remains a need in the art for improved methods for identifying HFOs in brain recording data.

### SUMMARY

The presently-disclosed subject matter meets some or all of the above-identified needs, as will become evident to those of ordinary skill in the art after a study of information provided in this document.

This Summary describes several embodiments of the presently-disclosed subject matter, and in many cases lists variations and permutations of these embodiments. This Summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently-disclosed subject matter, whether listed in this Summary or not. To avoid excessive repetition, this Summary does not list or suggest all possible combinations of such features.

Provided herein are methods for identifying high frequency oscillations (HFOs) in neural signals from the brain, which can include data obtained using intracranial as well as noninvasive (scalp) recordings. In some embodiments, the method includes detrending candidate HFO signals and then testing the detrended candidate HFO signals for (i) amplitude, (ii) rhythmicity, and/or (iii) ringing (referred to herein as “strikes”) to detect the HFOs. In some embodiments, the relevant signal property for each strike tested is compared against critical values specific to each event and not adjusted or optimized a priori using heuristics or labeled training samples. That is, as discussed herein, the significance of each strike property is uniquely determined by the characteristics of the individual signal to be tested.

The presently-disclosed subject matter further includes methods for monitoring a subject who has been diagnosed as having epilepsy. In some embodiments, the method involves using the method of identifying HFOs as described herein to obtain neural signals from the brain of the subject. The HFOs are identified at a first time point, and subsequently the HFOs are identified at a second time point. The method further involves comparing presentation of the HFOs at the first and second time points. In some embodiments, the method also involves identifying changes in presentation of HFOs.

The presently-disclosed subject matter further includes a method for predicting whether a subject has epilepsy and/or predicting a location in the brain of the subject that is associated with epilepsy. In some embodiments, the method involves identifying HFOs according to the method of claim 1, wherein the neural signals are obtained from the brain of the subject; and analyzing presentation of the HFOs. Presentation of HFOs is described further herein above. In some embodiments, the presentation of the HFOs includes a presence of HFOs above a predetermined threshold and/or a presence of HFOs in a determined location. In some embodiments, the method further includes determining a seizure onset zone and/or a specific region involved in generating seizures based upon the analyzed presentation of HFOs.

### BRIEF DESCRIPTION OF THE DRAWINGS

The presently-disclosed subject matter will be better understood, and features, aspects and advantages other than those set forth above will become apparent when consideration is given to the following detailed description thereof. Such detailed description makes reference to the following drawings, wherein:

FIG. 1 shows a three strikes algorithm for high frequency oscillation (HFO) detection. Stage 1: A relaxed amplitude criterion is employed to identify events of interest in the iEEG. Stage 2: Candidate signals are detrended to emphasize ripples in the residual signal. Stage 3: Criteria for ripple amplitude (X), rhythmicity (Y), and (the absence of) ringing artifact (Z), respectively, are applied to the residual and generate binary outcomes—or strikes (e.g., X=0, Y=1, Z=1)—for each event. Stage 4: Events with one or more strikes form subsets with specific properties identified by a triple, XYZ: for instance, 1YZ events are high amplitude (X=1) but may or may not satisfy Y or Z criteria; X11 events are rhythmic but free from ringing, etc.

FIG. 2 shows graphs illustrating HFO examples. Ripple (80-250 Hz; upper three panels) and fast ripple (200-500 Hz; lower three panels) HFOs riding on slower transients in the iEEG), as detected by the Staba algorithm with relaxed r.m.s. amplitude threshold. BPF=bandpass-filtered.

FIGS. 3A-C show graphs illustrating spline-based decomposition of HFO baseline. Example of (A) HFO without spike, (B) HFO with spike, and (C) spike without an HFO. Each panel shows: raw iEEG for the detected event (upper panel); BIC vs. the number of breaks in the piece-wise cubic spline (*=optimal number) (lower left); and the original detection, fitting signal generated by the model, and residual signal to be tested for ringing artifact (lower right).

FIGS. 4A-C show graphs illustrating application of HFO criteria. Example of an (A) HFO without a spike, (B) HFO with a spike, and (C) an spike without an HFO, tested using amplitude, rhythmicity, and ringing criteria. Candidate HFOs detected in Stage 1 (left column) are tested against event-specific thresholds for amplitude, rhythmicity, and ringing after baseline rejection using BPF (center column) and spline-based (right column) filters.

FIG. 5 shows a graph illustrating ringing filters. Amplitude response of conventional FIR bandpass and central difference (CD) filters.

FIG. 6 shows graphs illustrating event class statistics from algorithm Stage 1. Upper: Relative proportions of HFOs free of co-occurring spikes (Class C1a), HFOs with spikes (C1b), and spikes or other artifacts (C2) in each patient. Lower: Distribution over all subjects of C1 and C2 proportions (Left), and of C1a and C1b within C1 (Right).

FIG. 7 shows graphs illustrating outcomes produced by all possible combinations of one-strike HFO detectors. Upper panel: The proportion (equivalent to SE (sensitivity)) of true positive (TP) and false negative (FN) detections of spike-free HFOs (C1a) and HFOs with spikes (C1b), and the proportion (equivalent to SP (specificity)) of true negative (TN) and false positive (FP) detections of spikes (C2) for all possible combinations (1YZ, X1Z, XY1, 11Z, etc.) of one-strike detectors applied after detrending the iEEG using splines. Lower panel: Boxplot distributions (n=9 subjects) of detection metrics SE, SP, and PR (precision) for all HFOs versus spikes (C2) for each detector.

FIG. 8 shows graphs illustrating effect of iEEG detrending method on HFO detection. Boxplot distributions of detection metrics SE, SP, and PR across subjects (n=9) are shown for detector combinations 1YZ, 1Y1, and 111 applied to candidate HFO signals from Stage 1 detrended using BPFs (Left panels) and spline-based filters (Right panels). The BPF method detects HFOs with relatively high SE but poor SP and moderate PR. When spline-based detrending is used instead, SP and PR are appreciably elevated but with lower SE, particularly for 111 (three strikes). Combination 1Y1, which considers ripple amplitude and ringing, but ignores rhythmicity, provides moderate SE, high SP, and high PR.

FIG. 9 shows schematic examples of application of the three strikes HFO detector (111). Top panel: A pure HFO (Left), an HFO with a co-occurring spike (Center), and a pure spike (Right). Middle panel: Raw iEEG trace associated with detection (Red) shown with estimated baseline (Black) and residual (Blue) signals after baseline removal using splines. Lower panel: The residual of each event is tested using event-specific numerical criteria for adequate amplitude (Amp), rhythmicity (Rhyth), and the absence of ringing artifact (Ring) and the outcome compared against ground truth to quantify the performance of the HFO detector.

FIG. 10 shows a schematic example of application of the three strikes HFO detector.

FIG. 11 shows a cortical grid placement in patient B identifying seizure onset zone (SOZ). and actual resection margin (dark grey spots).

FIG. 12 includes (Left panel) an example of detection of ripple (80-250 Hz) oscillations riding on slower transients in the ECOG) using the relaxed Staba 2002 algorithm; and (Right panel) shows how to fit original signal by spline method and then measure number of peaks, rhythmicity, and Xcorr of residual signal. BPF: bandpass filtered.

FIG. 13 includes (Upper and left panel) shows an example of two-dimension real reference HFO map of the actual data of one patient. (upper and right panel) shows the actual HFO distribution across channels, and (lower panel) shows how the synthetic distribution changes along with different number of events for three samples of synthetic subsamples images.

FIG. 14 includes left and middle panels showing the spatial correlation distributions vs number of epochs per window for S1-4, and a right panel showing the mean correlation curves vs number of epochs per window at S1-4, where each curve represents the mean correlation for windows of that state. Note: spatial correlation was measured between windows containing different numbers of epochs and the reference image (RI).

FIGS. 15A and 15B include an example when the correlation trends at S1-4 were not different (FIG. 15A), and an example when the correlation trend at S1 was different from S2-4 (FIG. 15B). Upper panel shows the spatial correlation distribution vs number of events per channel at S1-4, lower and left panels show the pooled distribution trends of S1-4, and lower and right panels show the ecdf of correlation distribution of S1-S4.

FIG. 16 shows (left panel) the mean correlation curves of seven patients, and (right panel) the distribution of the number of events per channel corresponding with correlation=0.99.

FIG. 17 shows black scatter data represented the correlations of the synthic data, and grey scatter data represented the correlations of the actual data.

FIG. 18 shows the correlation distribution of the actual data when the threshold (number of events per HFO-channel=15).

While the disclosure is susceptible to various modifications and alternative forms, specific embodiments thereof have been shown by way of example in the drawings and are herein described below in detail. It should be understood, however, that the description of specific embodiments is not intended to limit the disclosure to cover all modifications, equivalents and alternatives falling within the spirit and scope of the disclosure as defined by the appended claims.

### DESCRIPTION OF EXEMPLARY EMBODIMENTS

The details of one or more embodiments of the presently-disclosed subject matter are set forth in this document. Modifications to embodiments described in this document, and other embodiments, will be evident to those of ordinary skill in the art after a study of the information provided in this document. The information provided in this document, and particularly the specific details of the described exemplary embodiments, is provided primarily for clearness of understanding and no unnecessary limitations are to be understood therefrom. In case of conflict, the specification of this document, including definitions, will control.

Provided herein are methods for identifying high frequency oscillations (HFOs) in neural signals from the brain, which can include data obtained using intracranial as well as noninvasive (scalp) recordings. The neural signal data can be obtained, for example, using magnetoencephalographic (MEG) or electroencephalographic (EEG). The neural signal data can also be obtained, for example, using electrocorticography (ECoG), intracranial electroencephalography (iEEG), and stereoelectroencephalography (SEEG). As will be appreciated by those of ordinary sill in the art, EEG refers to non-invasive measurement from the scalp. Subsequent variations on EEG have an added descriptor that differentiating the methodology, e.g., interacranial is the descriptor for iEEG, which is more invasive than the measurements taken from the scalp using EEG. As will be appreciated by one of ordinary skill in the art, HFOs range from about 80 Hz to about 500 Hz in frequency, and therefore it can be desirable to obtain neural signal data at a rate of about 1,000 samples/second (s/s). In epilepsy units, for example, it is common for EEG data to be recorded at a rate of at least about 500 s/s.

In some embodiments, the method includes detrending candidate HFO signals and then testing the detrended candidate HFO signals for (i) amplitude, (ii) rhythmicity, and/or (iii) ringing (referred to herein as “strikes”) to detect the HFOs. In some embodiments, the relevant signal property for each strike tested is compared against critical values specific to each event and not adjusted or optimized a priori using heuristics or labeled training samples. That is, as discussed in detail below, the significance of each strike property is uniquely determined by the characteristics of the individual signal to be tested.

Accordingly, in contrast to existing methods, the methods disclosed herein identify HFOs without any preset threshold, optimized or arbitrary, or associated assumption that one size fits all. In other words, as compared to other methods, the method of the presently disclosed subject matter does not use training data (i.e., no training on labeled or unlabeled sample data), does not use heuristic parameter thresholds/limits based on subjective human expertise, and does not employ assumptions that the data contain samples of both HFOs and non-HFOs. Rather, the presently-disclosed methods test any candidate events for one or more of three properties: amplitude, rhythmicity, and (the absence of) ringing. With consideration to these unique features, the methods disclosed herein are referred to as objective and training-free, which is described further herein.

The detrending of the candidate HFO signals eliminates the transient baseline and sifts out candidate HFO signals for further analysis through strike testing. In some embodiments, the detrending includes optionally prescreening the raw EEG data, such as intracranial EEG (iEEG), using a finite impulse response (FIR) filter. The iEEG includes unipolar, bipolar, or Laplacian potentials, for example, that are obtained by combining the individual iEEG signals from a set of electrodes. For example, in one embodiment, the raw EEG data is first divided into successive five-minute epochs of bipolar iEEG signals (in principle, the method is just as readily applied to referential/unipolar signals or other derivations, such as a Laplacian filter, obtained by combining multiple signals) which are bandpass-filtered from 80-500 Hz using a 20^{th} order FIR filter (fir1 in Matlab, designed with the impulse response constrained by a Hamming window, and applied forward and back to recover the linear phase shift in the output). Next, the root-mean-squared (rms) value is computed in a moving 3 ms window, and signal segments with successive rms values over 3 standard deviations (SDs) above the mean rms value of the five-minute baseline and at least 6 ms long are identified (events within 10 ms of each other may be merged). Finally, events with six or more peaks 3 SD above the mean of the rectified bandpass-filtered five-minute baseline signal are retained as candidate HFO signals. The FIR filtering above is basically the algorithm by Staba et al., [1] which has been modified with a more permissive rms threshold (3 SDs instead of 5 SDs above the mean) to admit any genuine HFOs buried in the iEEG while limiting candidates to a manageable number for visual confirmation.

While the prescreening FIR filter discussed above to emphasize high frequency activity effectively detrends the raw iEEG, it can introduce false ripples, an artifact commonly referred to as ringing. As such, in some embodiments, the candidate HFO signals identified through prescreening are subject to additional detrending. In some embodiments, the additional detrending of the candidate HFO signals includes a spline method, where piece-wise polynomials are fitted to the signal in order to eliminate the baseline and/or avoid the ringing artifact. For example, in one embodiment, the spline method includes dividing the raw iEEG into segments of equal length and then fitting each segment to a cubic spline (splinefit in Matlab) to preserve continuity of the model signal and its derivatives at the segment boundaries. The number of segments or breaks may be tuned to adjust the fit so that the model closely follows the baseline but passes through any ripples that might be associated with HFOs. In another embodiment, determining the preferred number of breaks required for each detection in order to suppress spikes and retain HFOs includes testing different break lengths, from 2 up to n points (the number of samples in the signal), to optimize the model fit. In a further embodiment, before fitting, 5 ms of signal before and after each detected event are included to preserve continuity at the boundaries.

In some embodiments, the fitting spline for each break is generated and subtracted from the raw signal to produce the residual. Bayes information criterion (BIC) [30], which balances conflicting terms representing the goodness-of-fit of the model and the number of degrees of freedom, respectively, is computed as a function of the number of breaks as follows:

$$BIC = - 2 log L + K log n . \qquad ( 1 )$$

Here, L is the joint likelihood that the samples of the residual belong to a normal probability model, n the number of samples in the signal, and K the number of degrees of freedom of the model based on the number of fitting parameters used. Since each cubic spline has four coefficients, K is four times the number of breaks. The number of breaks that gives the optimal BIC value is used to fit the candidate signal with the understanding that a break length of two points is not considered since it would simply pass through all points in the raw signal and give a zero residual. By modeling and removing spikes or other transients in the baseline as discussed above, HFOs are emphasized in the residual (raw signal minus baseline model).

Although discussed herein primarily with respect to prescreening first followed by additional detrending through the spline method, as will be appreciated by those skilled in the art, the disclosure is not so limited and may include other suitable methods of identifying candidate HFO signals and/or detrending prior to strike testing. For example, in some embodiments, the candidate HFO signals are identified through any other suitable prescreening method prior to additional detrending through the spline method. In another example, the candidate HFO signals identified by prescreening through linear filtering, such as the FIR method or any other suitable method, are subject to strike testing without the additional detrending through the spline method. In a further example, the raw EEG is detrended through the spline method directly without first being prescreened through linear filtering.

Once prescreened and/or detrended, the candidate HFO signals are strike tested to separate HFOs from spikes or other transients in the EEG data and identify true HFOs. The first strike test is the amplitude test, which includes deriving a critical amplitude threshold for each event and identifying positive peaks in the rectified residual based upon the derived threshold. In some embodiments, deriving the critical value for each event includes first padding the candidate HFO signal with two times its length of signal on either side. The residual from detrending is then rectified (e.g., by taking the absolute value so that all values are positive, including peaks), the signal peaks are located, and the peaks within the event are assigned positive labels (pos) and the peaks within the padded portions are assigned negative (neg) labels. Next, in contrast to existing methods that choose an arbitrary value for the amplitude threshold, such as Staba et al. [1] which uses 5 standard deviations (SDs) above the mean of a five-minute baseline, a specific threshold that best discriminates the pos and neg peak values for each event is determined by maximizing a chosen criterion that incorporates measures of sensitivity and precision associated with applying a threshold to predict the label of each peak: pos if above the threshold and neg if below. In one embodiment, this criterion may be an Fβ score, also known as F-score. The Fβ score is the harmonic mean of sensitivity and precision associated with prediction of the label of each peak as a function of a threshold. Parameter β is chosen to reflect the relative importance ascribed to sensitivity and precision in the Fβ score (e.g., β=1.0 would value them equally). Suitable β values include any positive value depending on the relative importance of sensitivity and precision in the application domain. For example, in one embodiment, β is set to ⅔ so that precision is valued 50% more than sensitivity: i.e., false positives (spurious HFO peaks) are less desirable than false negatives (missed HFO peaks). Other values such as 0.5 or 2 are possible. Alternative criteria to the F-beta score may also be used: for instance, the Fowlkes-Mallows index (square-root of the product of sensitivity and precision). If six or more pos peaks (equivalent to three complete waves, the bare minimum required to give the appearance of sustained oscillation), are identified as significant (i.e., above the critical threshold) in the rectified residual, the amplitude test is passed.

The second strike test is the rhythmicity test, where the Rayleigh Index (RI) of signal peaks is computed for the rectified residual to estimate the periodicity of signal peaks after detrending and compared with surrogate values of peak times generated for the same number of random peaks within the event interval. The RI is a widely used measure of phase clustering in

circular statistics [32]. To compute the RI, the mean frequency of peaks in the residual is first estimated as the reciprocal of the mean inter-peak interval:

$$f = \frac{n - 1}{{\sum }_{f = 1}^{n - 1} Δ {T}_{j}} \qquad ( 2 )$$

Here n is the number of peaks in the rectified residual, ΔTⱼ is the time between consecutive peaks, and Tⱼ is the time of each peak. Next, the angular location (phase) of each peak relative to the mean peak frequency f is expressed as a complex phasor:

$${r}_{j} = {e}^{i 2 π {fT}_{j}} \qquad ( 3 )$$

where i is the complex identity, \sqrt{−1}. Then the mean phasor amplitude over all peaks gives the Rayleigh Index:

$$RI = \vert \frac{1}{n} {\sum }_{j} {r}_{j} \vert . \qquad ( 4 )$$

To test for significant rhythmicity, RI is compared with any suitable number of surrogate values generated for the same number n of random peak times within the event interval. By design, these peaks will be at random phase relative to their mean frequency f and therefore reflect aperiodic or arrhythmic behavior. A suitable number of surrogate values is chosen to test for the probability that a value as high as RI could have occurred by chance. For instance, if 19 surrogates are used, when the true RI exceeds the largest of the surrogate values the test event is deemed significantly rhythmic at the 95% confidence level (i.e., 19 in 20); another way of saying that there is only a 5% (1 in 20) chance that such a high value of RI could have occurred. Thus, RI is effectively compared with a threshold that is unique to each event. In other embodiments, alternate tests for significant rhythmicity, RI can be used, such as, for example, computing the Fourier transform of the signal segment, locating the peak amplitude in the frequency domain, and testing its value against surrogates generated by randomly shuffling the samples of the signal and reestimating this peak.

The third strike test is the test for ringing artifact, where a cross-correlation between the outputs of the FIR filter and a reference filter is examined to assess whether the FIR filter is introducing a ringing artifact. The reference filter includes any suitable filter that will not produce ringing. In one embodiment, for example, a time domain filter with minimal ringing but relaxed frequency selectivity (broad passband) is used as the reference to determine whether the FIR filter, such as the 80-500 Hz FIR filter (BPF) discussed in connection with detrending above, produces ringing in response to a particular event. In another embodiment, this reference filter is a linear phase central difference (CD) operator (i.e., yₖ=xₖ₊₁−xₖ₋₁); in effect, a bandpass filter but with less steep transitions and without sharply defined cutoff frequencies. Without wishing to be bound by theory, it is expected that the outputs of the two filters will be strongly correlated (with at most a phase delay) if the input is an HFO without a sharp transient. In contrast, it is expected that they will be uncorrelated if the input is a spike, because the FIR filter will produce ringing while the CD filter will not. That is, the correlation is expected to be high for spike-free HFOs, intermediate-to-high for HFOs co-occurring with spikes, and low for spikes alone. The CD filter is particularly of interest since for a sinusoidal input, it will preserve the morphology of the signal and only introduce a phase delay, thus making the output strongly correlated with the input signal. However, the use of other reference filters—for instance an FIR filter with a broader pass band (e.g., 10-800 Hz) than 80-500 Hz instead of the CD filter, or subtracting a spline fit with no intermediate breakpoints from the original segment of signal—may also reduce ringing and allow the same cross-correlation criterion to be applied to test for the presence of the ringing artifact.

In some embodiments, the correlation between the FIR and reference filter outputs is estimated as the peak cross-correlation, r, between the outputs after allowing for phase differences produced by filtering. To determine whether r is significant, it is compared against Tr, the largest of a number of surrogate values generated by correlating the CD output with randomly shuffled residual signals. Any suitable number of surrogate values may be generated. This number is fixed based on the confidence with which we would like to say that the value of r is significant and therefore an indication of true correlation. For instance, in some embodiments if 19 surrogates are used and r is greater than the largest of them (Tr), the correlation is deemed significantly greater than chance with a 5% probability; if 99 surrogate values are used, that probability is 1%, and the correlation is deemed significant at the 99% confidence level. Any other suitable confidence level based upon the number of surrogates generated can be employed.

Any suitable combination of strike tests disclosed herein may be employed to identify HFOs in the candidate HFO signal. In some embodiments, identifying the HFOs includes testing the candidate HFO signal with a single strike test. In some embodiments, identifying the HFOs includes testing the candidate HFO signal with a combination of any two strike tests. In some embodiments, identifying the HFOs includes testing the candidate HFO signal with all three strike tests. The specific combinations are referred to herein using a three-character string, XYZ, where X represents the amplitude strike test, Y represents the rhythmicity strike test, and Z represents the ringing strike test. For example, 1Y1 refers to events that satisfy amplitude and ringing criteria-but without regard to rhythmicity; X1Z events are significantly rhythmic, but without consideration of their amplitude or possible ringing effects; and 111 events satisfy all three criteria.

In some embodiments, the number and type of strikes tested will impact the proportion of true HFOs admitted (referred to herein as “sensitivity” or “SE”), the proportion of non-HFOs rejected (referred to herein as “specificity” or “SP”), and the proportion of detections that are true HFOs (referred to herein as “precision” or “PR”). For example, in one embodiment, of the three individual tests, XY1 provides the highest SE (e.g., over 80%) and PR for true HFOs with highly variable SP; while X1Z and 1YZ provide lower numbers for SE, SP, and PR. In another embodiment, combining two of three individual tests increases SP (e.g., over 70%) but decreases SE, as compared to the one-strike tests, without significantly changing PR, although the specific embodiment of 1Y1 provides high SP (over 80%) and high PR (over 70%) with a less precipitous drop in SE (a median of 50%). In a further embodiment, combining all three tests further increases SP (e.g., over 90%) while reducing PR slightly (e.g., over 60%) and SE more sharply (e.g., below 25%). Although including the rhythmicity strike test may reduce the sensitivity, making a distinction between rhythmic and non-rhythmic HFOs may have diagnostic implications given the claims that rhythmic events are more likely to be correlated with epileptogenicity. Accordingly, in some embodiments, the method includes combining all three strike tests to identify HFOs with high specificity and precision. Additionally or alternatively, in some embodiments, the method includes combining the amplitude and ringing strike tests to provide slightly lower specificity with increased precision and sensitivity.

Since the strike tests according to one or more of the embodiments disclosed herein set unique thresholds for each individual event using resampling statistics, the methods disclosed herein including such strike tests are completely objective, require no prior training or tuning, and may be implemented to work in an objective and training-free manner. Additionally, the methods disclosed herein are capable of separating events into not only HFOs (class C1) and spikes (class C2), but also HFOs without spikes (C1a) and HFOs with spikes (C1b). Thus, the methods disclosed herein facilitate the identification of HFOs that co-occur with spikes, a subset that is believed to have special significance.

The presently-disclosed subject matter further includes methods for monitoring a subject who has been diagnosed as having epilepsy. In some embodiments, the method involves using the method of identifying HFOs as described herein to obtain neural signals from the brain of the subject. The HFOs are identified at a first time point, and subsequently the HFOs are identified at a second time point. The method further involves comparing presentation of the HFOs at the first and second time points. In some embodiments, the method also involves identifying changes in presentation of HFOs.

As will be appreciated by one of ordinary skill in the art upon study of this document, the presentation of HFOs can include, for example, timing in relation to spikes, rhythmic discharges, or other markers of epilepsy. Presentation of HFOs can also include the amplitude, frequency or spectral content, rhythmicity, duration or other characteristics of HFOs.

The presentation of HFOs can be useful, for example, to predict distinctions/distinguish between normal from pathological brain tissue. For example, in some instances, a location in the brain that has HFOs co-occurring with spikes can be pathological while another location that has HFOs without spikes (or spikes without HFOs) is normal (i.e., does not generate seizures). Additionally, changes in the presentation of HFOs can indicate a change in the location or specific region(s) in the brain involved in generating seizures in the subject, or can indicate that there is a change, such as a reduction or an increase, in HFOs identified in the neural signals from the brain, i.e., neural signal data. Such changes in presentation of HFOs can be associated with a change in the severity and/or presence of epilepsy.

Furthermore, such changes in presentation of HFOs can be associated with the efficacy of a treatment and/or a rationale for maintaining or changing the treatment. For example, if the presentation of HFOs indicates a change in the location in the brain involved in generating seizures, for a subject who is a good candidate for neuromodulation or stimulation treatments, one can target the newly-identified location of the brain for neuromodulation or stimulation. For another example, changes in presentation of HFOs could present a rationale for titrating the timing and or dose of treatments and interventions, including, for example, medication, stimulation, diet, or sleep. Changes in presentation of HFOs, such as a significant reduction or elimination of HFOs could also be the basis for reducing and/or discontinuing treatment, being mindful of reducing any unnecessary treatment that could have undesirable associated side effects.

In this regard, in some embodiments of the method for monitoring the subject, the second time point at which HFOs are identified is after receiving treatment for epilepsy. And in some embodiments, the first time point is prior to receiving treatment and/or is associated with a distinct treatment. In this regard, the method can further involve determining treatment efficacy, and maintaining or adjusting treatment, based on changes in presentation of HFOs. The treatment could be, for example, vagus nerve stimulation (VNS), responsive neurostimulation (RNS), deep brain stimulation (DBS), trigeminal nerve stimulation (TNS), anti-seizure medication, sleep medication, medical cannabis, ketogenic diet, surgery, or combinations thereof. Examples of anti-seizure medication that could be used include Carbamazepine (Tegretol), Valproic Acid (Depakote), Lamotrigine (Lamictal), Levetiracetam (Keppra), Oxcarbazepine (Trileptal), Topiramate (Topamax), Phenobarbital (Luminal), Phenytoin (Dilantin), and Ethosuximide (Zarontin). When a subject has been identified as having drug-resistant epilepsy (DRE), which is also known as refractory epilepsy or pharmacoresistant epilepsy, and the treatment the subject receives could involve a neuromodulation or stimulation treatment, or another treatment that excludes anti-seizure medication. Neuromodulation or stimulation treatments can include, for example, VNS, RNS, DBS, TNS, or combinations thereof.

The presently-disclosed subject matter further includes a method for predicting whether a subject has epilepsy and/or predicting a location in the brain of the subject that is associated with epilepsy. In some embodiments, the method involves identifying HFOs according to the method of claim 1, wherein the neural signals are obtained from the brain of the subject; and analyzing presentation of the HFOs. Presentation of HFOs is described further herein above. In some embodiments, the presentation of the HFOs includes a presence of HFOs above a predetermined threshold and/or a presence of HFOs in a determined location.

In some embodiments, the method further includes providing treatment when the subject is predicted to have epilepsy. The treatment can include, for example, VNS, RNS, DBS, TNS, anti-seizure medication, sleep medication, medical cannabis, and ketogenic diet.

In some embodiments, the method further includes determining a seizure onset zone and/or a specific region involved in generating seizures based upon the analyzed presentation of HFOs. In this regard, the method can further include targeting the onset zone and/or specific region with neuromodulation or stimulation, such as VNS, RNS, DBS, or TNS. In some embodiments, the method can further involve determining whether the onset zone and/or specific region is a candidate for surgical intervention.

As will be appreciated by one of ordinary skill in the art, methods disclosed herein that relate to predicting the presence of epilepsy or making a diagnosis can be used in some cases in combination with other methods. Such other methods that can be optionally used in combination with the presently-disclosed methods include, for example, diagnostic imaging methods, such as positron emission tomography (PET) and/or magnetic resonance imaging (MRI), video assessment, neuropsychological evaluation, and/or other EEG markers, including, but not limited to, spikes, sharps, rhythmic discharges, and seizures. As will be appreciated upon study of this document, in some embodiments, it may be useful to combine methods as disclosed herein with other methods to provide further assistance, for example, in distinguishing epileptogenic from normal brain tissue, which can be useful in the context of targeting onset zone and/or specific region with the brain of a subject.

Unless defined otherwise, all technical and scientific terms used herein have the same meaning as commonly understood by one of ordinary skill in the art to which the disclosure belongs. Any methods and materials similar to or equivalent to those described herein can be used in the practice or testing of the present disclosure, including the methods and materials are described below.

Following long-standing patent law convention, the terms “a,” “an,” and “the” refer to “one or more” when used in this application, including the claims. Thus, for example, reference to “a cell” includes a plurality of cells, and so forth.

Unless otherwise indicated, all numbers expressing quantities of ingredients,

properties such as reaction conditions, and so forth used in the specification and claims are to be understood as being modified in all instances by the term “about.” Accordingly, unless indicated to the contrary, the numerical parameters set forth in this specification and claims are approximations that can vary depending upon the desired properties sought to be obtained by the presently-disclosed subject matter.

As used herein, the term “about,” when referring to a value or to an amount of mass, weight, time, volume, concentration, percentage, or the like is meant to encompass variations of in some embodiments ±50%, in some embodiments ±40%, in some embodiments ±30%, in some embodiments ±20%, in some embodiments ±10%, in some embodiments ±5%, in some embodiments ±1%, in some embodiments ±0.5%, in some embodiments ±0.1%, in some embodiments ±0.01%, and in some embodiments ±0.001% from the specified amount, as such variations are appropriate to perform the disclosed method.

As used herein, ranges can be expressed as from “about” one particular value, and/or to “about” another particular value. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

The terms “comprising,” “including,” and “having” are intended to be inclusive and mean that there may be additional elements other than the listed elements.

As described hereinabove, the presently disclosed subject matter is directed to detection of HFOs that is “objective and training free,” in that it is not trained on either labeled or unlabeled sample data, nor does it encode heuristic parameter thresholds/limits based on subjective human expertise, nor does it assume that the data contain samples of both HFOs and non-HFOs. Rather, the presently-disclosed methods test any candidate events for one or more of three properties: amplitude, rhythmicity, and (the absence of) ringing. Objective and training free detection is distinct from and excludes supervised detection. Supervised approaches are modeled and optimized on limited, labeled sample/training data and/or use heuristic parameters and criteria based on expert knowledge or intuition that have been tweaked or tuned to perform in the desired manner. Accordingly, supervised approaches can require labeled sample/training data and fitting models to the data, seeing differences between positive and negative samples. Distinctly, objective and training-free detection in accordance with the methods disclosed herein do not require labeled sample/training data nor use of heuristic approaches, which encode arbitrary criteria based on expert experience, which implies foreknowledge of the properties and their typical values of HFOs. Objective and training free detection is also distinguished from unsupervised detection, to the extent that certain methods that have been referred to as unsupervised, such as clustering, either require training data, or assume that the training data (or data to be modeled) contain samples of both HFOs and non-HFOs. Certain unsupervised methods work with unlabeled sample/training data, but do assume that the sample/training data contain both HFOs and non-HFOs. Distinctly, objective and training-free detection in accordance with the methods disclosed herein do not require training data and do not make this assumption about samples containing both HFOs and non-HFOs.

As used herein, the terms “optional” or “optionally” means that the subsequently described event or circumstance can or cannot occur, and that the description includes instances where said event or circumstance occurs and instances where it does not.

As used herein, the term “subject” can be a vertebrate, such as a mammal, a fish, a bird, a reptile, or an amphibian. Thus, the subject of the herein disclosed methods can be a human, non-human primate, horse, pig, rabbit, dog, sheep, goat, cow, cat, guinea pig or rodent. The term does not denote a particular age or sex. Thus, adult and newborn subjects, as well as fetuses, whether male or female, are intended to be covered.

All combinations of method or process steps as used herein can be performed in any order, unless otherwise specified or clearly implied to the contrary by the context in which the referenced combination is made.

The presently-disclosed subject matter is further illustrated by the following specific but non-limiting examples. The following examples may include compilations of data that are representative of data gathered at various times during the course of development and experimentation related to the presently-disclosed subject matter. Those skilled in the art will recognize, or be able to ascertain, using no more than routine experimentation, numerous equivalents to the specific substances and procedures described herein.

### EXAMPLES

Spikes and other sharp transients observed in the intracranial EEG (iEEG) can confound the detection of high frequency oscillations (HFOs), which may have demonstrated diagnostic value in epilepsy surgical evaluation. When spikes are passed through linear digital filters—commonly used in HFO analysis to emphasize the tell-tale ripples—“ringing” artifacts that masquerade as HFOs can occur. As such, there remains a need for intuitive, objective, and universal criteria for identifying and characterizing HFOs, which may play a critical role in the surgical management of epilepsy. These Examples present an objective and training-free classification scheme to effectively distinguish HFOs from spikes without requiring labelled or unlabeled training data. It is hoped that the approach described here helps build consensus as to what constitutes genuine HFO activity

In order to accomplish this, overnight, seizure-free iEEG recordings from nine epilepsy patients admitted for invasive presurgical evaluation were identified and used for analysis with informed consent. Separate criteria for HFO amplitude, rhythmicity, and ringing, were specified and applied to candidate events after detrending their iEEG signals with splines to eliminate sharp transients in the baseline without introducing a ringing artifact. Bootstrap methods were used to test whether each individual event satisfied the criteria; hence, no supervision was required to tune parameters or thresholds.

Using a proposed “three strikes” algorithm, genuine HFOs were identified with high specificity (95%) and precision (84%), but poor sensitivity (16%). Leaving out the rhythmicity criterion greatly improved detection sensitivity (51%) without compromising specificity (89%) and precision (88%). Spline-based detrending increased the specificity of HFO detection compared to conventional 80-500 Hz linear bandpass filtering, but reduced the sensitivity.

### Example 1

This Example discusses an objective and training-free framework for HFO analysis that relies on intuitive criteria without parameter tuning or arbitrary thresholds to identify and separate HFOs from spikes. It is relatively simple and capable of fast, high-channel-count analysis. The utility of this approach is demonstrated in overnight iEEG recordings from human epilepsy patients undergoing invasive evaluation for epilepsy surgery.

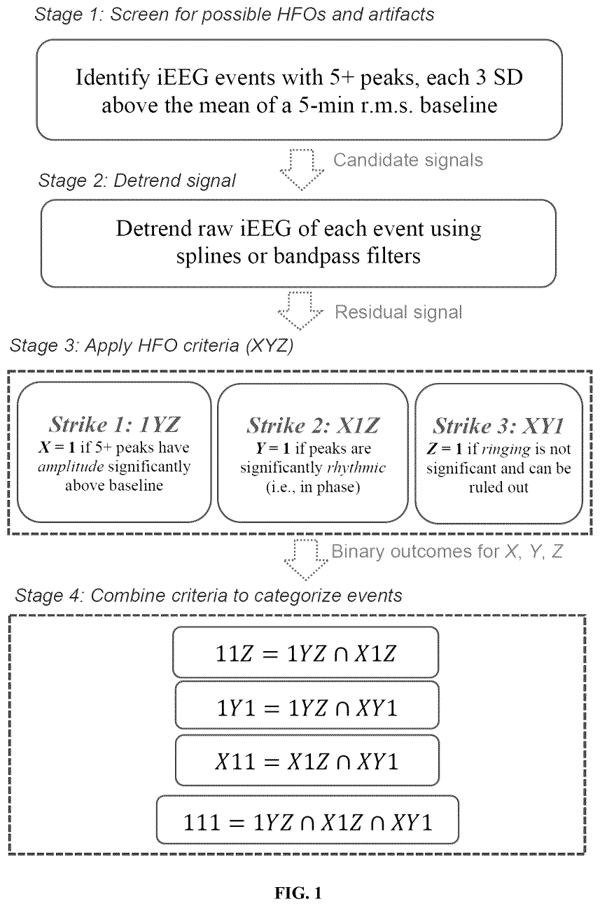

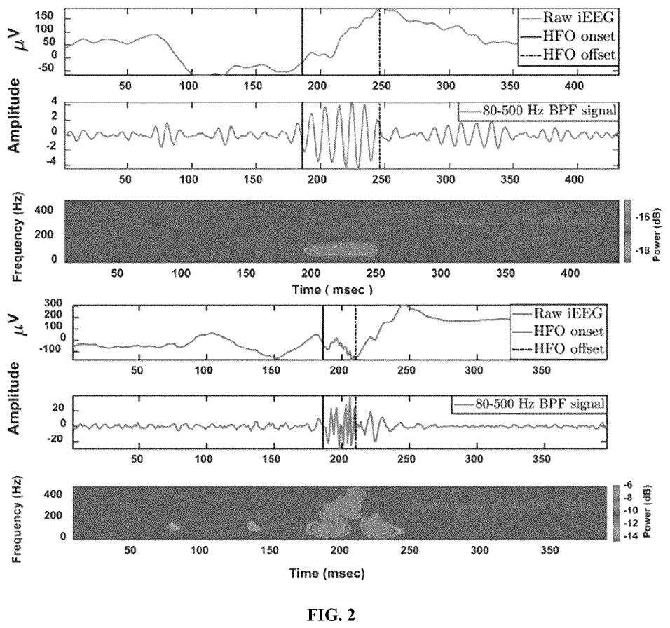

The method can be described with reference to the flowchart in FIG. 1, which illustrates an exemplary three strikes algorithm for HFO detection. In Stage 1, a relaxed amplitude criterion is employed to identify events of interest in the iEEG. The Staba criterion is used in this depicted example. In other embodiments, another prescreening method and/or other brain recording data capable of detecting HFOs could be employed. In Stage 2, candidate signals are detrended to emphasize ripples in the residual signal. In Stage 3, criteria for ripple amplitude (X), rhythmicity (Y), and (the absence of) ringing artifact (Z), respectively, are applied to the residual and generate binary outcomes—or strikes (e.g., X=0, Y=1, Z=1)—for each event. In Stage 4, events with one or more strikes form subsets with specific properties identified by a triple, XYZ: for instance, 1YZ events are high amplitude (X=1) but may or may not satisfy Y or Z criteria; X11 events are rhythmic but free from ringing, etc. Events with three strikes (i.e., 111) are most likely to be true HFOs and can be struck from the baseline. At the same time, different sub-types of events can convey different information to the diagnostic process. For instance, 101 denotes non-rhythmic HFOs while 111 denotes rhythmic HFOs and these could correlate with different things.

### Methods

### Data Acquisition and Event Labeling

Continuous iEEG recordings were acquired with IRB approval and informed consent from nine patients with pharmacorefractory epilepsy undergoing invasive presurgical evaluation, five at the University of Texas Southwestern Medical Center (labeled A-E) and four at the University of Kentucky (labeled P1-P4). iEEG signals were sampled at 1000 Hz (except for E, which was sampled at 2000 Hz but down-sampled to 1000 Hz for consistency with the others). The number of iEEG contacts in each case ranged from 40 to 57 and the duration of interictal (i.e., seizure-free) data from 4 to 12 hours (details in Table 1). To generate validation data for testing the HFO detection algorithm, the algorithm by Staba et al., [1] which is highly sensitive to HFO activity but limited in specificity (see Stage 1 below for further detail), was applied with a relaxed amplitude threshold to iEEG channels within the physician-marked seizure onset zone (SOZ) referenced to their immediate neighbors (a bipolar montage). This was done to gather a mixed sample of true HFOs and other transients or artifacts and is also regarded as Stage 1 of the proposed algorithm, in which a wide net is cast for HFO candidates to be screened further.

TABLE 1
Summary information on iEEG data used for analysis.

| | | | | | | Three channels |
|---|---|---|---|---|---|---|
| Patient | No. of | No. of SOZ | | | | with the most |
| ID | channels | channels | SOZ* location | Electrode type | Montage | detections |
| A | 60 | 10 | Temporal and | Grid and strip | TG1-20, OF1-6, | TG16-17, |
| | | | occipital lobes | | OB1-6, TF1-4, | 17-18, and |
| | | | | | OA1-6, OC1-6, | 18-19 |
| | | | | | TB1-4, TU1-4, | |
| | | | | | and TP1-4 | |
| B | 64 | 8 | Temporal lobe | Grid | LG 1-64 | LG 41-42, |
| | | | | | | 57-58, and |
| | | | | | | 58-59 |
| C | 64 | 13 | Frontal and | Grid | LG 1-64 | LG1-2, 18-19, |
| | | | parietal lobes | | | and 51-52 |
| D | 48 | 14 | Frontal, | Grid | LG 1-48 | LG 31-32, |
| | | | parietal, and | | | 39-40, 46-47 |
| | | | temporal lobes | | | |
| E | 66 | 5 | Subtemporal | Grid and strip | LG 1-48, PT 1- | MT 1-2, AT |
| | | | and lateral | | 6, AT 1-6, and | 2-3, and 3-4 |
| | | | temporal lobe | | MT 1-6 | |
| P1 | 32 | 2 | Frontal lobe | Grid, strip, | LFG 4-8, 12-16, | LFG 13-14, |
| | | | | and depth | 20-24, 28-31, | LID 1-2, |
| | | | | | LOS 2-6, and | and 2-3 |
| | | | | | LID 1-6 | |
| P2 | 48 | 2 | Parietal lobe | Depth | L and R AH 1-8, | LPH 1-2, 2-3, |
| | | | | | L and R MH 1-8, | and 3-4 |
| | | | | | L and R PH 1-8 | |
| P3 | 76 | 20 | Temporal and | Grid; strip | RFT 1-64, RSA | RSP 1-2, |
| | | | frontal lobe | | 1-6, and RSP | RFT 13-14, |
| | | | | | 1-6 | and 14-15 |
| P4 | 60 | 3 | Temporal lobe | Grid; strip | RTG 1-48, | RTG 44-45, |
| | | | | | ATS 1-6, and | 45-46, and |
| | | | | | PTS 1-6 | 46-47 |

*SOZ = Seizure onset zone as determined by neurologist.

The first 300 detections from the three SOZ channels with the highest apparent HFO activity were selected, reviewed visually, and labeled as HFOs, HFOs with co-occurring spikes, or spikes/artifacts. Quasirhythmic events with at least three oscillations that formed concentrated “blobs” in the time-frequency spectrogram were considered HFOs (two examples are shown in FIG. 2) provided the raw signal did not appear to contain sharp artifacts that may have morphed into ripples when filtered. The majority of verifiable events detected from grid and depth electrodes by this screening process belonged to the category of ripples (80-250 Hz) and fast ripples (>250 Hz), respectively, while the rest were electrographic spikes or artifacts. These detections made up the algorithm validation set. All data analysis was performed on Matlab™ (Mathworks, Natick, MA).

### Stage 1. Prescreen for Candidate HFOs

The HFO detector is composed of three distinct stages. In the first of these, a superset of events is gathered by employing the algorithm by Staba et al., [1] slightly modified to admit any genuine HFOs buried in the iEEG while limiting candidates to a manageable number for visual confirmation. In each recording, successive five-minute epochs of bipolar iEEG signals were bandpass-filtered from 80-500 Hz using a 20^{th} order FIR filter (fir1 in Matlab, designed with the impulse response constrained by a Hamming window, and applied forward and back to recover the linear phase shift in the output). The root-mean-squared (rms) value was computed in a moving 3 ms window and signal segments with successive rms values over 3 standard deviations (SDs) above the mean rms value of the five-minute baseline and at least 6 ms long were identified; events within 10 ms of each other were merged. Finally, events with six or more peaks 3 SD above the mean of the rectified bandpass-filtered five-minute baseline signal were retained for further reckoning in Stage 2. This is in essence the Staba algorithm, except with a more permissive rms threshold (3 SDs instead of 5 SDs above the mean).

### Stage 2. Detrend Candidate HFO Signals

The FIR filter used in Stage 1 to emphasize high frequency activity effectively detrends the raw iEEG, but can introduce false ripples, an artifact commonly referred to as ringing. To avoid this artifact, an alternative method was developed for iEEG detrending in which cubic splines are used to model and eliminate the transient baseline and sift out HFO activity where present for further analysis. This procedure was applied to the candidate iEEG segments from Stage 1 and the results compared against bandpass filtering.

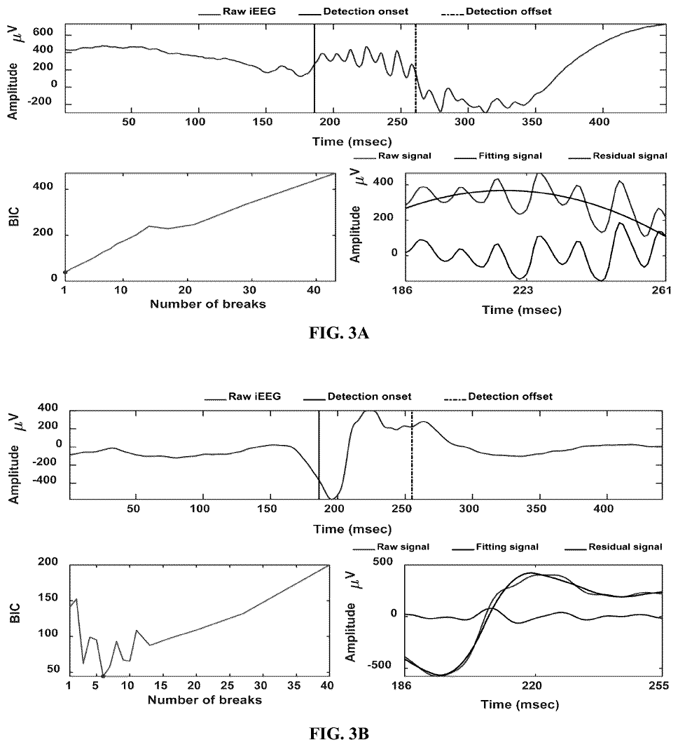

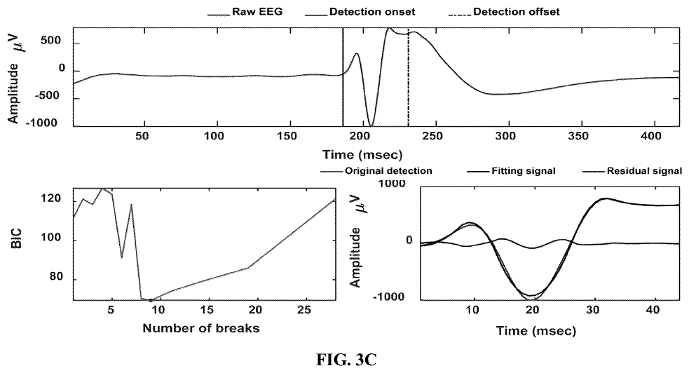

In the spline method, the raw iEEG is divided into segments of equal length and each segment is fitted to a cubic spline (splinefit in Matlab) to preserve continuity of the model signal and its derivatives at the segment boundaries. The number of segments or breaks is tuned to optimize the fit, so that the model closely follows the baseline but passes through any ripples that might be associated with HFOs. By modeling and removing spikes or other transients in the baseline, HFOs are emphasized in the residual (raw signal minus baseline model). FIGS. 3A-C give examples in which an HFO without a spike, an HFO with a spike, and a spike without an HFO, respectively, are detrended in this manner. The challenge is to determine the optimal number of breaks required for each detection so that the residual suppresses spikes and retains HFOs. Before fitting, 5 ms of signal before and after each detected event are included to preserve continuity at the boundaries. Different break lengths, from 2 up to n points (the number of samples in the signal) are tested to optimize the model fit.

The fitting spline for each break is generated and subtracted from the raw signal to produce the residual. Bayes information criterion (BIC) [30], which balances conflicting terms representing the goodness-of-fit of the model and the number of degrees of freedom, respectively, is computed as a function of the number of breaks as follows:

$$BIC = - 2 log L + K log n . \qquad ( 1 )$$

Here, L is the joint likelihood that the samples of the residual belong to a normal probability model, n the number of samples in the signal, and K the number of degrees of freedom of the model based on the number of fitting parameters used. [31] Since each cubic spline has four coefficients, K is four multiplied by the number of breaks. The number of breaks that gave the lowest BIC value was used to fit the candidate signal. Naturally, a break length of two points was not considered since it would simply pass through all points in the raw signal and give a zero residual.

### Stage 3. Apply Specific HFO Criteria

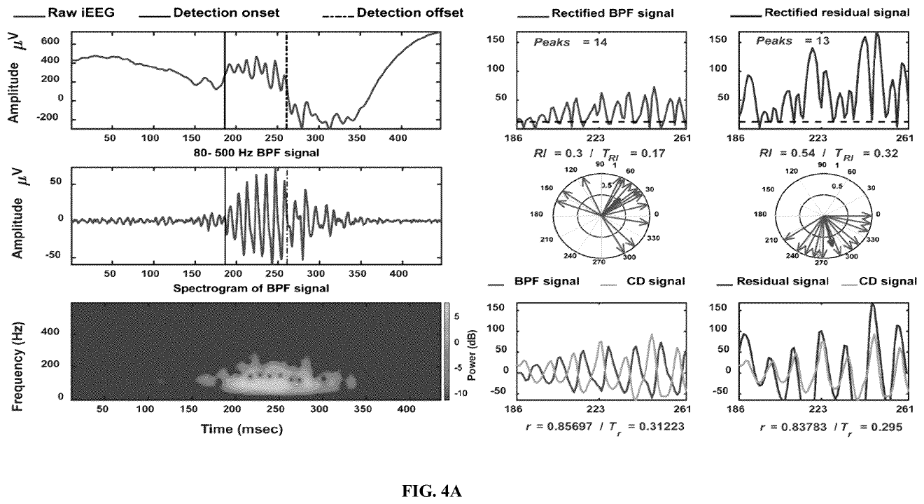

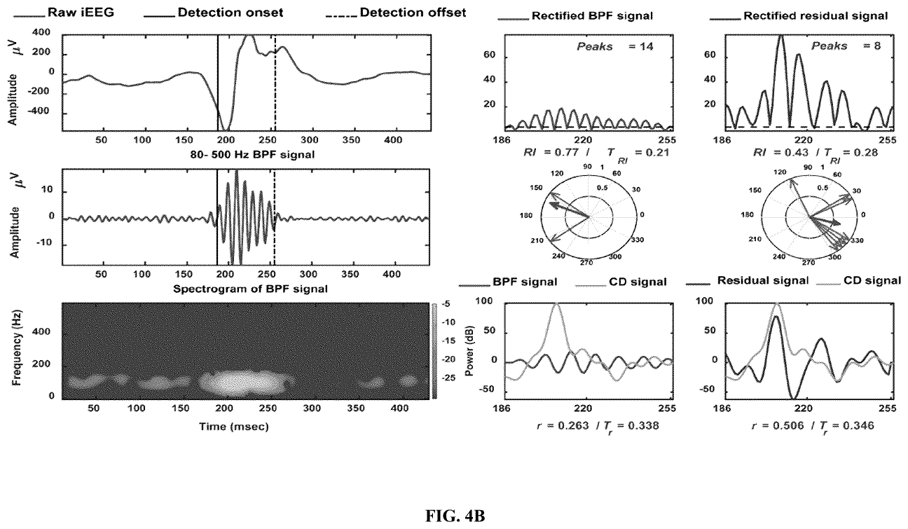

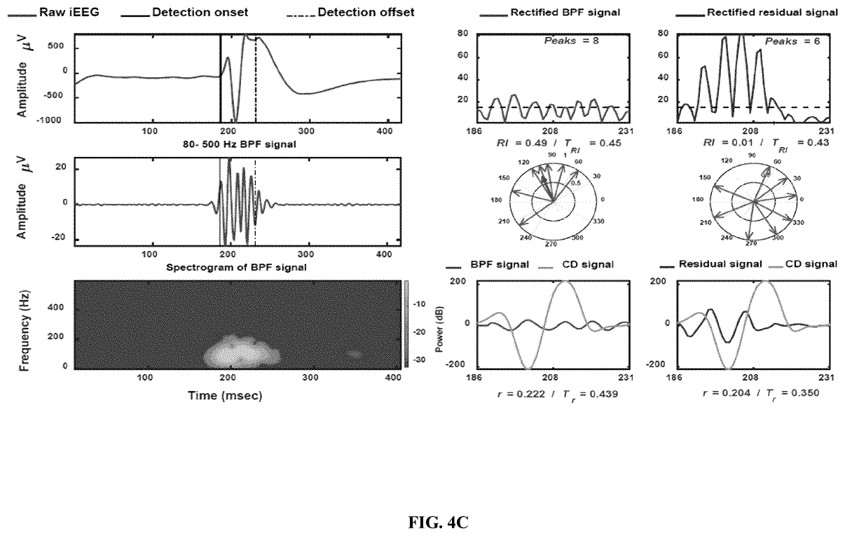

Criteria for amplitude, rhythmicity, and the presence of ringing artifact in the residual—the three strikes, respectively—were devised to separate HFOs from spikes or other transients in the iEEG. In each test, the relevant signal property is compared against critical values specific to each event and not adjusted or optimized a priori using heuristics or labeled/unlabeled training samples (see FIGS. 4A-C for examples).

### Strike 1: The Amplitude Test

The amplitude criterion is satisfied if six or more peaks—equivalent to three complete waves, the bare minimum required to give the appearance of sustained oscillation—in the rectified residual exceed a critical amplitude threshold. Rather than choose an arbitrary value for this threshold—for instance, Staba et al [1] use 5 standard deviations above the mean of a five-minute baseline as an amplitude threshold (see Stage 1 above)—a critical value specific to each event is derived here as follows. The detected event from Stage 1 is first padded with two times its length of signal on either side. Then the residual from either bandpass or spline filtering is rectified and the signal peaks located. The peaks within the event and padded portions are assigned positive (pos) and negative (neg) labels, respectively. A threshold that best discriminates the pos and neg peak values is determined by maximizing an Fβ score, which is the harmonic mean of sensitivity and precision associated with applying a threshold to predict the label of each peak: pos if above the threshold and neg if below. The choice of parameter β reflects the relative importance ascribed to sensitivity and precision in the Fβ score. While β=1.0 would value them equally, β was set to ⅔ so that precision is valued 50% more than sensitivity: i.e., false positives (spurious HFO peaks) are less desirable than false negatives (missed HFO peaks). If six or more pos peaks are identified (three complete oscillations) in the rectified residual, the amplitude test is passed (see FIGS. 4A-C for examples).

### Strike 2: The Rhythmicity Test

The Rayleigh index, a widely used measure of phase clustering in circular statistics [32] is computed for the rectified residual to estimate the periodicity of signal peaks after detrending. The mean frequency of peaks in the residual is first estimated as the reciprocal of the mean inter-peak interval:

$$f = \frac{n - 1}{{\sum }_{f = 1}^{n - 1} Δ {T}_{j}} \qquad ( 2 )$$

Here n is the number of peaks in the rectified residual, ΔTⱼ is the time between consecutive peaks, and Tⱼ is the time of each peak. Next, the angular location (phase) of each peak relative to the mean peak frequency f is expressed as a complex phasor:

$${r}_{j} = {e}^{i 2 π {fT}_{j}} \qquad ( 3 )$$

where i is the complex identity, \sqrt{−1}. Then the mean phasor amplitude over all peaks gives the Rayleigh Index:

$$RI = \vert \frac{1}{n} {\sum }_{j} {r}_{j} \vert \qquad ( 4 )$$

To test for significant rhythmicity, RI is compared with 19 surrogate values generated for n random peak times within the event interval. By design, these peaks will be at random phase relative to their mean frequency f and therefore reflect aperiodic or arrhythmic behavior. If the true RI exceeds the largest of the surrogates the test event is deemed significantly rhythmic at the 95% confidence level. Thus, RI is effectively compared with a threshold that is unique to each event and therefore does not rely on training data or experience (see FIGS. 4A-C for examples).

### Strike 3: Test for Ringing Artifact

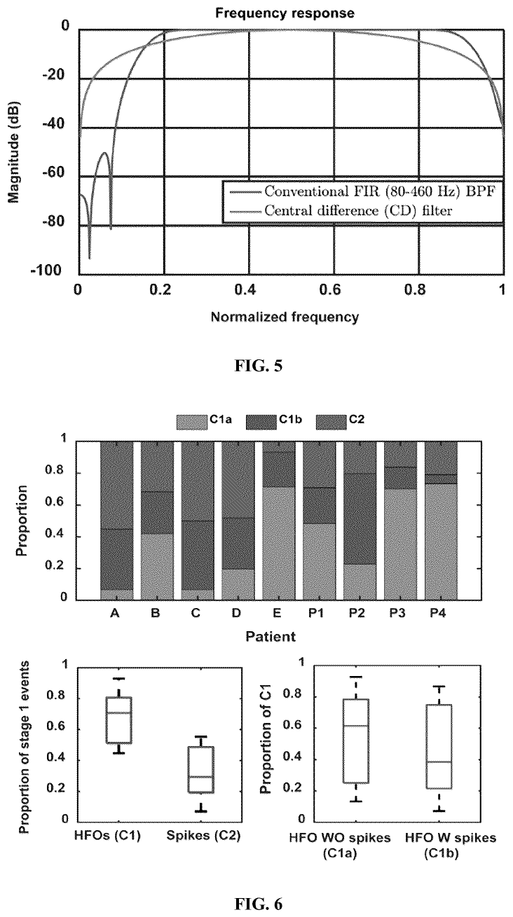

The ringing effect, also called the Gibbs phenomenon, occurs when a steep signal passes through a filter. Indeed, the strength of the effect depends on the characteristics of the filter. The ideal bandpass filter is rectangular in the frequency domain, but a sinc function in the time domain, characterized by decaying sinusoidal oscillations, or ripples. In the time domain, convolution between a steep signal and the sinc function therefore produces an oscillatory response with artificial ripples: this artifact is known as ringing. Here, a time domain filter with minimal ringing but relaxed frequency selectivity (broad passband) was used as a reference for determining whether the 80-500 Hz FIR filter (BPF) used in Stage 1 produces ringing in response to a particular event. This reference filter is a linear phase central difference (CD) operator (i.e., yₖ=xₖ₊₁−xₖ₋₁); in effect, a bandpass filter but with less steep transitions and without sharply defined cutoff frequencies (see FIG. 5). The cross-correlation between the outputs of the FIR and CD filters is examined to assess whether the former is corrupted by a ringing artifact.

It is expected that the outputs of the two filters will be strongly correlated (with at most a phase delay) if the input is an HFO—in the ideal case, a sinusoidal input—without a sharp transient. In contrast, they will be uncorrelated if the input is a spike, because the FIR filter will produce ringing while the CD filter will not; even with splines, a ringing-like artifact can be generated at times if the spline fails to closely track the transient baseline. Theoretically, the correlation would be high for spike-free HFOs, intermediate-to-high for HFOs co-occurring with spikes, and low for spikes alone.

The correlation between FIR and CD filter outputs was estimated as the peak cross-correlation, r, between the outputs after allowing for phase differences produced by filtering. To determine whether r was significant, it was compared against Tr, the largest of 99 surrogate values generated by correlating the CD output with randomly shuffled residual signals. If r>Tr, the correlation is deemed significant at the 99% confidence level (see FIGS. 4A-C for examples).

### Stage 4. Three Strikes: Combine Multiple Criteria to Detect and Characterize HFOs

In Stage 3, the events were put in the superset of candidates from Stage 1—after baseline removal via BPF or spline filtering in Stage 2—through three tests: for amplitude, rhythmicity and ringing. Each test derives event-specific feature thresholds to separate candidates into two classes: 1. HFOs; and 2. Spikes or other transients. Succumbing to each test constitutes a strike (in sporting parlance).

Events that satisfy all three criteria are most likely to be HFOs and have struck out of the baseline, in a manner of speaking. However, strikes can be combined in seven different ways: the first three are simply the individual criteria, and the rest are combinations of any two and finally all three criteria. Each combination is referred to by a three-character string, XYZ, with X, Y, or Z set to 1—a strike—if the amplitude, rhythmicity, or ringing criterion is satisfied, respectively; or left as X, Y, or Z if the criterion is not applied. For instance, 1Y1 refers to events that satisfy amplitude and ringing criteria-but without regard to rhythmicity; X1Z events are significantly rhythmic, but without consideration of their amplitude or possible ringing effects; and 111 events satisfy all three criteria. The 111 test is the most stringent or conservative of the seven combinations, and events captured thus lie at the intersection of groups that satisfy either amplitude, rhythmicity, or ringing criteria.

### Performance Evaluation

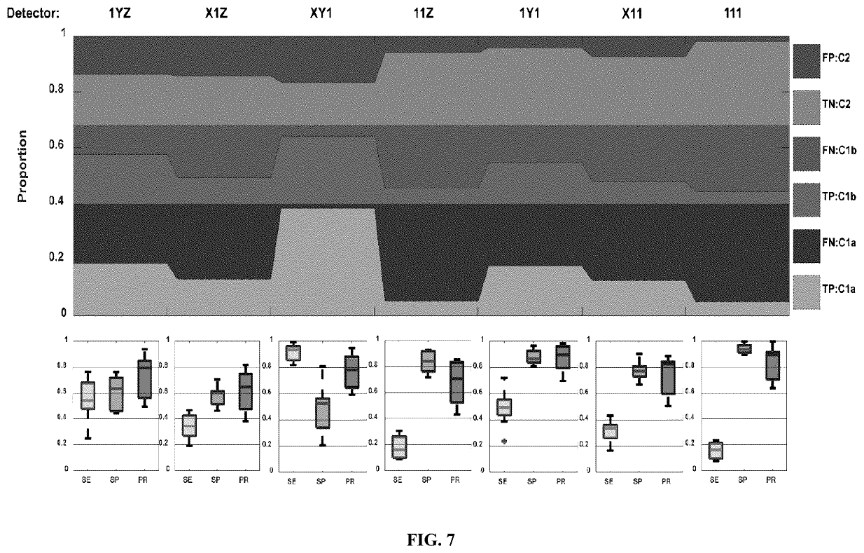

To assess and compare the performance of the strike detectors, conventional statistical measures were employed: 1. Sensitivity (SE), the proportion of true HFOs admitted; 2. Specificity (SP), the proportion of non-HFOs rejected; and 3. Precision (PR), the proportion of detections that are true HFOs. The main purpose of these detectors is to separate events into two classes: HFOs (C1) and spikes or other transients (C2). However, HFOs can occur with or without spikes (see FIG. 6). In fact, HFOs that co-occur with spikes could have special significance. One embodiment could involve the use of the HFO detector alongside a separate spike detector to identify intersecting events. Hence, the ability of the detectors to differentiate true HFOs (class C1) in the Stage 1 superset into HFOs without spikes (C1a) and HFOs with spikes (C1b) was further studied by computing metrics SE, SP, and PR for these subclasses as well (see FIG. 7, upper panel).

### Results

### Gross Statistics of Stage 1 Events

For each of the nine recordings, the first 300 events—a total of 2700—gathered in Stage 1 of the classification algorithm from the three most active channels were visually reviewed and labeled according to their properties as C1a (spike-free HFOs), C1b (HFOs with co-occurring spikes), or C2 (spikes or other artifacts). HFOs of both ripple (80-250 Hz) and fast ripple (250-500 Hz) categories were observed, though these differences were not quantified further.

The proportion of C1 events (i.e., true HFOs) in each recording varied widely (roughly 40-80%), as did the proportion of C1a and C1b within C1 (about 10-90%); these distributions are presented in FIG. 6. The purpose of this step is to identify all possible HFOs in that interval while limiting the number of detections to a manageable number for manual review. If the algorithm were terminated at this point, the HFO yield would have an SE of close to 100% and PR of 40-80%. Further lowering the peak threshold (set at 3 SD over the mean of the rectified baseline) would merely reduce PR further and increase the number and proportion of C2 events to be screened in the later stages of analysis.

### Performance of HFO Classifiers

The amplitude, rhythmicity, and ringing criteria were applied individually and in combination with each other to the spline-detrended signals of candidate events from Stage 1 to generate seven sets of detection outcomes (results in FIG. 7).

One-strike detection: Of the three individual tests, XY1 gave the highest SE (over 80%) and PR for C1 but highly variable SP; while X1Z and 1YZ gave low to middling numbers for SE, SP, and PR. In general, the number of peaks with significant amplitude was relatively high for HFOs and low for spikes. Some spikes that were poorly detrended by the spline method were admitted by XY1 as false positives, which accounted for its moderate SP. Rhythmicity of the peaks was less distinctive for HFOs compared to spikes after detrending. However, in the ringing test the cross-correlation (r) between the detrended signal and CD-filtered raw signal was high for HFOs and low for spikes. These effects are illustrated in FIGS. 3A-C and 4A-C.

Two-strike detection: When any two of three individual tests were combined, SP increased in comparison to the one-strike tests to over 70%, but without a noticeable change in SP and at the cost of SE. 1Y1 was the exception and gave high SP (over 80%) and high PR (over 70%) with a less precipitous drop in SE (a median of 50%).

Three-strike detection: Combining all three tests further increased SP to over 90% with a slight reduction in PR to over 60%; however, SE dropped sharply to below 25%.

The results above suggest that combining the amplitude, rhythmicity, and ringing criteria—the 111 detector—captures HFOs with high SP and moderate PR at the cost of greatly reduced SE. When the rhythmicity criterion is ignored—in effect the 1Y1 detector—SE is much improved without sacrificing SP and PR.

Trends in SE and SP across detectors were very similar whether spike-free HFOs (C1a) or HFOs co-occurring with spikes (C1b) were considered (FIG. 7, upper panel). This indicates that the criteria for amplitude, rhythmicity, and ringing are not biased in either direction by the presence or absence of spikes or other transients in the baseline.

### Effect of Detrending: Splines Versus BPF

The analysis presented above was repeated using BPFs instead of splines in Stage 2 to detrend candidate HFO signals from Stage 1. The resulting trends in SE, SP, and PR across detectors were very similar, but in each case SE was higher, PR slightly lower, and SP much lower. The differences are illustrated in FIG. 8 for three detectors: 1YZ (only amplitude tested), 1Y1 (amplitude and ringing tested) and 111 (amplitude, ringing, and rhythmicity tested).

When BPFs were employed, the residual signals appeared quasirhythmic for real HFOs as well as spikes (see FIGS. 4A-C). For the spline-based detrending, however, it was easier to distinguish between HFOs and spikes on this basis depending on the goodness of fit in each instance (see FIGS. 3A-C). In general, the BIC criterion proposed to optimize the number of spline segments (breaks) needed to fit a signal appeared to produce a favorable compromise between parsimony and accuracy. The number of breaks was relatively low for spike-free HFOs (see FIG. 3A) and the fitting signal passes through the ripples while removing the slower trend in the baseline. If an HFO was close to a spike the number of breaks set by the BIC was greater in order to follow the trail of the spike while passing through the ripples (see FIG. 3B). Finally, a relatively large number of breaks was specified for spikes without HFOs in order to capture their trajectory albeit with some artificial ripples in the residual (see FIG. 3C).

### Discussion

The cost of error associated with diagnostic mapping for epilepsy surgery is high. The use of HFO analysis for this purpose is attractive for the prospect of analyzing a brief interictal baseline as opposed to waiting an unknowable number of days for seizures to emerge. On the other hand, the small amplitude and timescale of these events make them hard to label with absolute certainty; furthermore, visual review to sort HFOs from artifacts and other phenomena demands significant time and labor. The features of this problem domain make it ripe for automation. And not surprisingly, there is already a vast and fast-growing body of literature on computerized HFO analysis. HFO detectors or classifiers fall into one of two categories.

The first category—Type I—is trained offline on positive and negative samples of previously collected data sets to fix its parameters, and then applied to a patient's data to identify HFOs with similar properties. The training may be supervised or unsupervised: if supervised, the labels are known and explicitly incorporated into the training process [20, 21, 23]. The models and features employed can be quite complex, but not necessarily. In fact, previous work demonstrates that a simple logistic regression model trained on five HFO features—density, connectivity, amplitude, log power, and peak frequency—successfully differentiates between

HFOs detected in locations inside and outside the SOZ for out-of-sample patient data [22]. If unsupervised, the labels are not known a priori but natural partitions or clusters in the training data set are identified using statistical or algorithmic criteria [28, 29] under the assumption that one or more clusters correspond to HFOs. Once trained, the classifier can be used to predict labels for a patient's data, either offline or in real time; it need not be assumed that there are HFOs in the data.

Type II classifiers encode expert knowledge or heuristics into numerical rules or signal processing steps to be applied to any patient's data to label events as HFO or non-HFO [1, 24, 26]. These rules may include parameter values or thresholds that have been set arbitrarily or fine-tuned based on experience with previous data—for instance, the 99th percentile of the r.m.s. of a five-minute baseline, or perhaps five standard deviations above the mean—to behave in the desired manner. The algorithm by Staba et al [1] may be regarded as the progenitor of many of these detectors, since similar rules and criteria (e.g., bandpass filtering, r.m.s.-based peak threshold, etc.) may have been used, as a first step or with some modification. In some instances, the thresholds are further optimized on existing data [16, 25, 27].

Whether Type I methods, which are optimized on specific data sets, will generalize to other data is hard to know without substantial validation. While the appeal of these methods lies in the power of machine learning to discriminate between different labeled or unlabeled classes, the complexity of the features and “black-box” behavior are, ipso facto, hard to interpret and evaluate in physically meaningful terms. Type II methods are similar to Type I in that they have been optimized on previous data—except by human experience rather than numerical modeling. They are attractive from the perspective that the desired HFO properties are more directly encoded in the signal processing pipeline, and therefore easier to interpret and debate as to their validity.

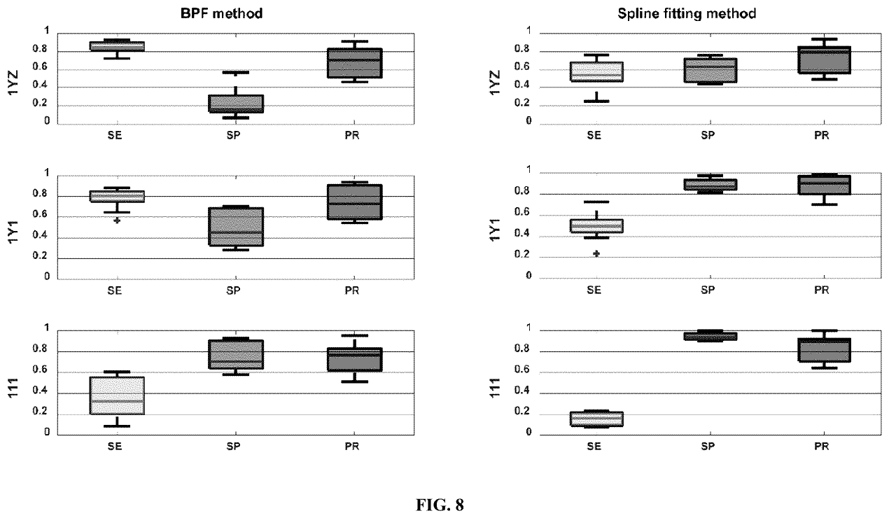

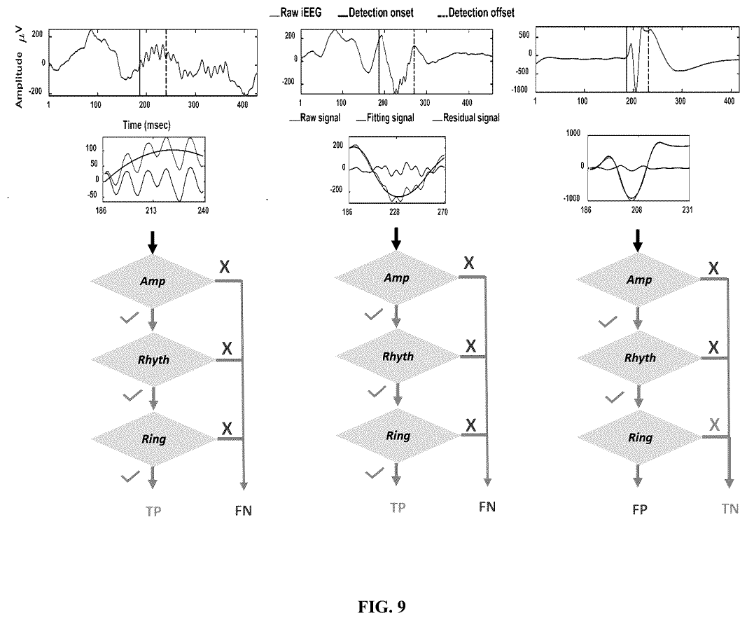

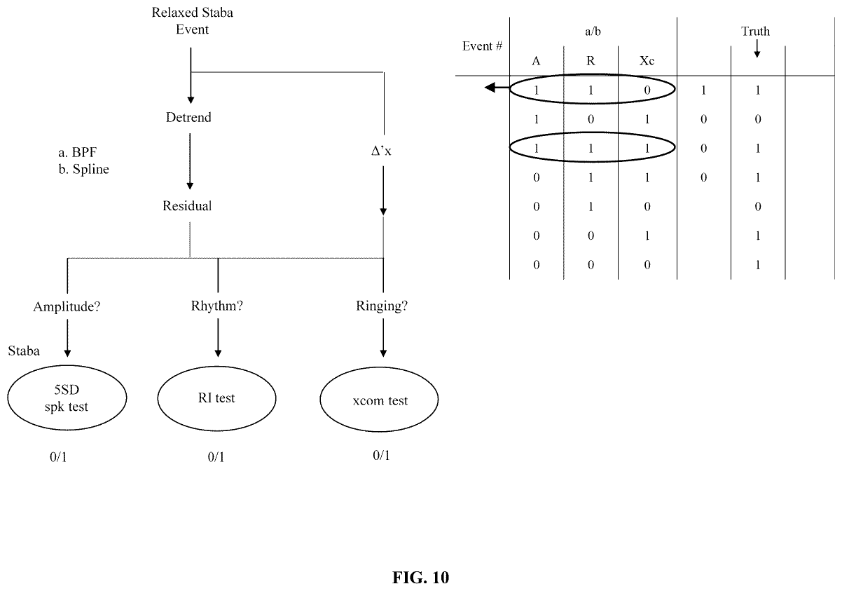

In view thereof, without making judgement on the relative accuracy of these existing classifiers, which can vary widely and are dependent on the criteria used in the annotation, a distinction is made in the way that they distinguish HFOs from other events. More specifically, in the present study, for the proposed three strikes algorithm, events must satisfy three simple criteria—for amplitude, rhythmicity, and ringing—to be considered genuine HFOs. Signals, detrended either using BPFs or splines, are deemed to represent HFOs if they contain oscillations that swing too high above the baseline and are too rhythmic for chance to have played a role, and if a ringing artifact can be ruled out as the source of these ripple-like oscillations; illustrative example of this process are given in FIGS. 9-10. However, the significance of each property is uniquely determined by the characteristics of the individual signal to be tested. There is no preset threshold, optimized or arbitrary, and with it the assumption that one size fits all. Bootstrap procedures are used to determine whether amplitude, rhythmicity, and ringing criteria are satisfied without the need for a reference sample or external validation. By comparing the events sorted thus against manually assigned truth labels, it was found that combining all three criteria gives high SP and PR, albeit with limited SE, which is a relatively minor concern. Interestingly, ignoring Strike 2, the rhythmicity criterion, greatly improves SE without a drop in PR (see FIG. 8). Making a distinction between rhythmic and non-rhythmic HFOs may have diagnostic implications, however, given the claims that rhythmic events are more likely to be correlated with epileptogenicity [33].

Stage 1, in which a relaxed form of the Staba 2002 algorithm is employed to gather candidates for screening, is not essential to this scheme but chosen merely for convenience. Any other means could have been used to identify events of interest, whether artifact or not.

In Stage 2, the signal is detrended to emphasize the tiny ripples associated with HFO activity before applying the three strikes. A BPF is a convenient and powerful means for removing low-frequency trends from a signal that are typically much larger in amplitude than HFOs are can mask their presence. Unfortunately, sending a signal through a BPF not only unmasks an HFO (if present) but introduces false ripples—the ringing artifact—if the event contains a spike or other sharp transient with a broad spectral presence. This phenomenon is guarded against at the front end by specifying an alternative method of detrending based on cubic splines that alleviates ringing, and at the back end by introducing a simple test for the presence of ringing.

The spline-based detrending method uses a criterion (BIC) that balances the goodness-of-fit of the model to the data against the number of segments (parameters) required to achieve this fit. This compels the model to follow the large amplitude trend while ignoring and thus passing through the small amplitude ripples where present. The number of breaks determined by minimizing the BIC turn out to be small for spike-free HFOs, moderate for HFOs co-occurring with spikes depending on their relative size, and large for spikes without HFOs (see examples in FIGS. 3A-C). In general, detrending with splines gave lower SE but higher SP and PR than with BPFs (see FIG. 8). If the fit of the spline model to the signal is poor, or if a BPF is used instead, the prospect of false ripples arises and the ringing test (Strike 3) provides added protection.

### Conclusion

Despite the proliferation of methods, there remains a need for intuitive, objective, and universal criteria for identifying and characterizing HFOs, which may play a critical role in the surgical management of epilepsy. Discussed herein is a simple Three Strikes algorithm that tests candidate signals for significant amplitude, rhythmicity, and ringing—without the need for predetermined thresholds—and thus evaluates their “HFO-ness.” A spline-based method was also designed to detrend signals of interest with a much lower risk of introducing the ringing associated with BPFs. These processes in combination appear to identify HFOs with high specificity and precision. Without wishing to be bound by theory, it is believed that the approach described here may help build consensus as to what constitutes genuine HFO activity and the qualities that have the most diagnostic value in the surgical treatment of epilepsy.

### Example 2

This Example relates to effect of vigilance state and sample size on the spatial profile of HFOs.

### Data Acquisition

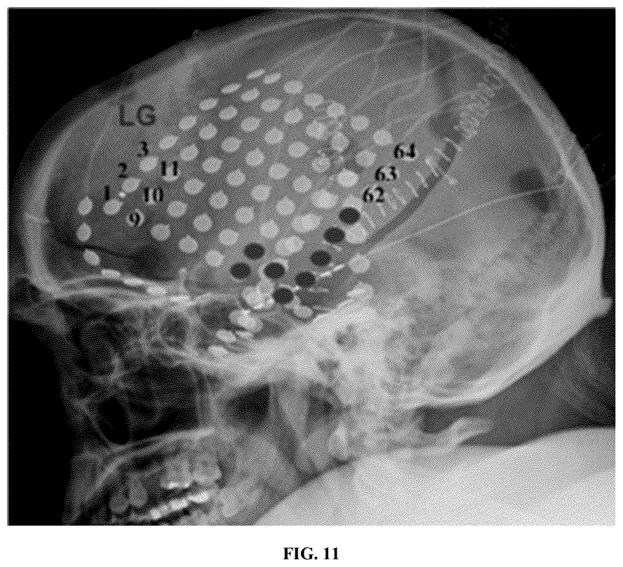

Nine epilepsy patients admitted for invasive presurgical evaluation, identified as A, B, C, D, E, P1, P2, P3, and P4, were monitored using ECoG (example of ECOG grid placement shown in FIG. 11). The data were sampled at 1000 Hz in subjects A-D and P1-P4 and 2000 Hz in subject E. Frontocentral and centro-occipital scalp EEG, horizontal EOG (only in five patients (A-E), submental EMG, and EKG were simultaneously recorded to help verify vigilance state. Here the bipolar derivation of the two ECoG contacts closest to fronto-central locations was used to determine vigilance state. The number of ECOG contacts used in this study ranged from 48 to 66 in each patient. All analysis was performed retrospectively using Matlab™ (Mathworks, Natick, MA). Patient P1 and P2 were excluded because they had very frequent seizures and limited interictal data.

### Automatic Detection and Verification of HFOs

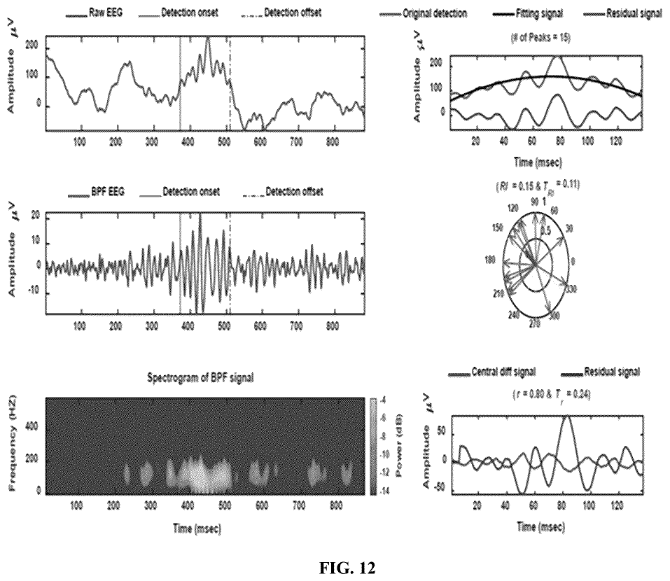

This method started with a relaxed amplitude threshold in the Staba algorithm to catch a superset of events for further analysis. Each consecutive 5-minute bipolar ECoG signals was bandpass-filtered (80-500 Hz) using a Hamming-window based FIR digital bandpass filter with 40th-order (applied forward and backward to recover linear phase shift). The root-mean-squared (rms) value was computed in a moving 3 ms window for each epoch. Successive values more than three standard deviations above the mean and >6 ms long were flagged and events with fewer than 6 peaks (each 3 SDs above mean) in the rectified filtered signal were rejected. To maximize the chances of getting genuine HFOs the “111” detector with the spline detrending method was applied to screen each candidate event because this detector would provide very high specificity and acceptable sensitivity. In this method, each event had to satisfy three different criteria related to amplitude, rhythmicity, and ringing artifact respectively. All detected events were visually verified. Quasirhythmic time-delimited events with at least three oscillations that formed concentrated “blobs” in the time-frequency spectrogram were deemed to be HFOs (one sample is shown in FIG. 12) provided the raw signal did not appear to contain sharp artifacts that may have produced ripples when filtered. Usually, verifiable events detected from the grids and depth electrodes by the “111” detector belonged to the category of ripples (80-250 Hz). fast ripples (>250 Hz), and a few spikes or other artifacts respectively.

### When to Sample HFOs?

To study the effect of vigilance state on the spatial HFO distribution and choose an appropriate spatial HFO profile for recording and mapping the HFO zone, the grid and strip channels were rearranged into a two-dimensional array or image. The entire recording of each patient was divided into 30-s sampling epochs in which the density of HFOs (the number of events per epoch) across channels was computed. In addition, a vigilance state (S1-S4) determined by the sleep scoring algorithm was assigned to each epoch. To balance the data, an equal number of epochs were randomly selected from states S1-S4; this number was set based on the minimum number of epochs available in any state for that patient. By averaging HFO density over 50% of this balanced set of epochs for each channel across states a reference map of HFO activity was generated under the assumption—or null hypothesis—that a HFO map (image) sampled from finite data in any vigilance state would be asymptotically identical to the reference image.

To measure the degree of similarity between any two HFO images, the Pearson correlation coefficient was computed between S1-S4 windows comprised of different numbers of consecutive epochs, but without replacement (for instance, the first set of S1 windows have one S1 epoch per window, the second set of S1 windows have two consecutive S1 epochs per window, etc.) and the reference image. In fact, the effects of two factors on similarity of a sample window with the reference window were studied: 1) the number of epochs per sample window (as a means of implicitly increasing the number of events per epoch); and 2) the number of events per HFO-channel measured for all sample windows. Where HFO-channel refers to channels on which at least one HFO is detected.

Therefore, these two factors would help to study the behavior of the similarity trend at S1-4 in terms of convergence, growth rate, variation, as well as whether the spatial HFO profile was fundamentally different among S1-S4. Based on these measured variables, a useful state to sample HFOs can be identified.

### How Many HFOs are Needed?

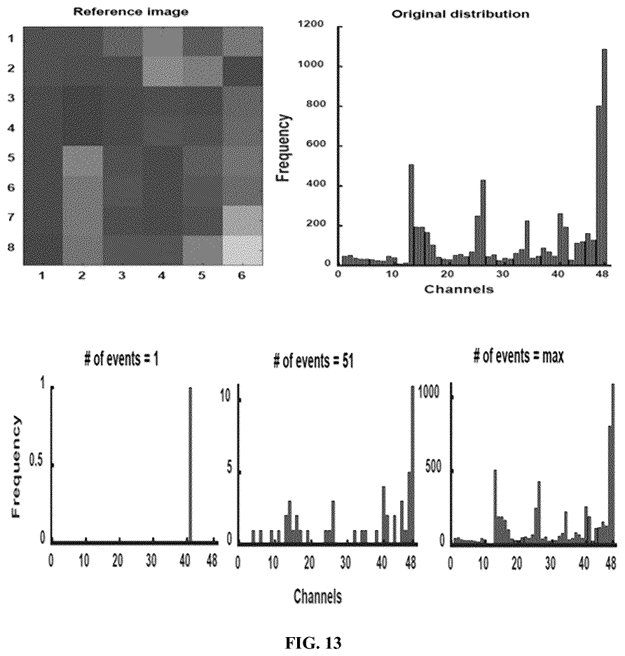

Studies were conducted to determine how large an HFO sample is adequate to reliably define the HFO zone. No previous study has attempted to formally answer this question; instead, most arbitrarily use one or more fixed 10-minute segments of ECOG, usually during slow wave sleep, for analysis. Here, the number of events per HFO-channel was used as a measure of sample size and the correlation of the associated spatial HFO map with the reference image was studied for different window sizes. As a reminder, a sampling window is defined here as an integer number of 30-second epochs. Therefore, there is no control over the number of HFOs in any window, only the number of epochs. Hence it is unlikely in practice that all possible values of HFOs per channel in a recording will be represented, which is the independent variable in the analysis. To generate regularly spaced data, HFOs per channel were divided into ten equally sized bins over the observed range of values and the distribution of correlation between each HFO image within each bin and the reference image computed. Here, the distribution of the reference HFO image was used for each patient, and based on that, synthetic subsample two-dimensional images were created. These synthetic subsample images have to follow the main reference image distribution and started with a random selection of one event per HFO-channel up to the maximum number of events per HFO-channel of that distribution (see FIG. 13). The similarity trend using the spatial correlation between these subsample images and the reference images was measured. Theoretically, we attempted to determine the minimum number of events per HFO-channel that gives a spatial HFO distribution that is essentially identical to the reference image, for instance, a correlation coefficient of 0.99 or greater between the two. That number would be used as a natural threshold on the number of events per HFO-channel. However, to apply that threshold in practice, it is first necessary to verify that the synthetic and actual spatial correlation trends are not statistically different. Therefore, the synthetic and actual correlations (pooled data from S1-S4) were divided into ten equal bins over the range of values observed, and the mean in each bin was measured. Finally, a paired t-test was applied to test for statistically significant differences between them.

### Results

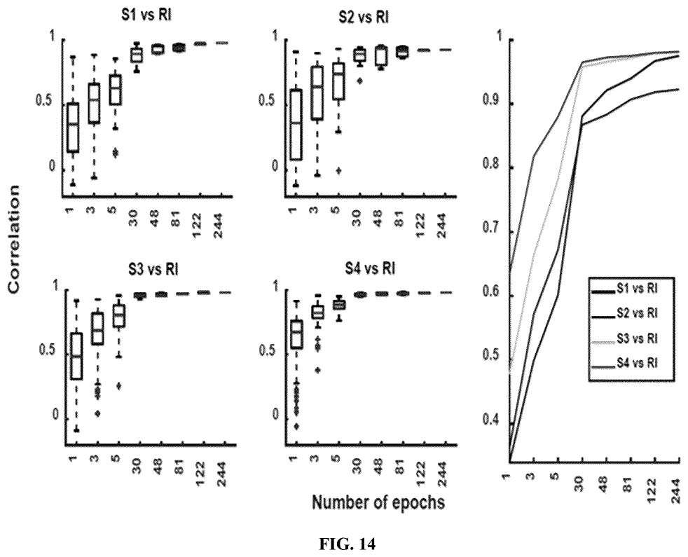

The spatial HFO pattern was studied in this analysis to see whether it is consistent and unique to each vigilance state. Two-dimensional arrays representing the spatial pattern of HFO activity of 30-sec S1-S4 epochs were created. The spatial correlation (similarity) between the sample window and the overall reference image as a function of both the number of epochs and number of events per HFO-channel measured at each sample window for a pooled 50% of S1-S4 epochs, is studied here to answer the questions of when to record and how many HFO samples constitute a reliable sample? FIG. 14 shows the behavior of the spatial correlation versus the number of epochs selected per sample window for one patient. It is noticed that the correlation distribution gradually increases in the mean, which asymptotically approaches unity as the number of epochs of S1-S4 increase (see FIG. 14, left panel). Also, the variance (spread) of the correlation distribution is highest at the minimum number of epochs per window and progressively decreases until it became zero at the maximum number of epochs per window. The mean correlation curves for S1-S4 were created by computing the mean of the correlation distributions, as shown in FIG. 14, right panel. In general, this shows that the rate of convergence of the correlation curve to unity is greatest for S4 and decreases progressively from S4 to S1.

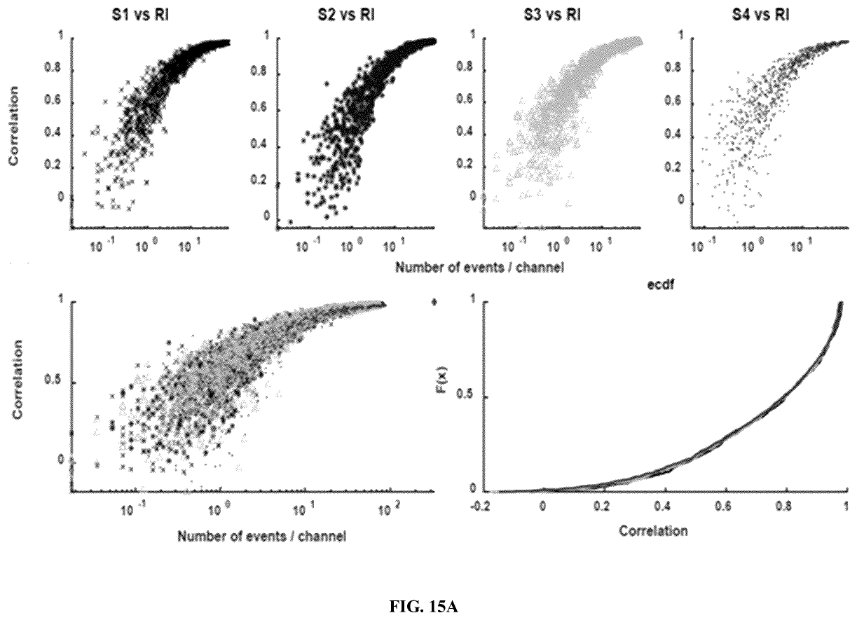

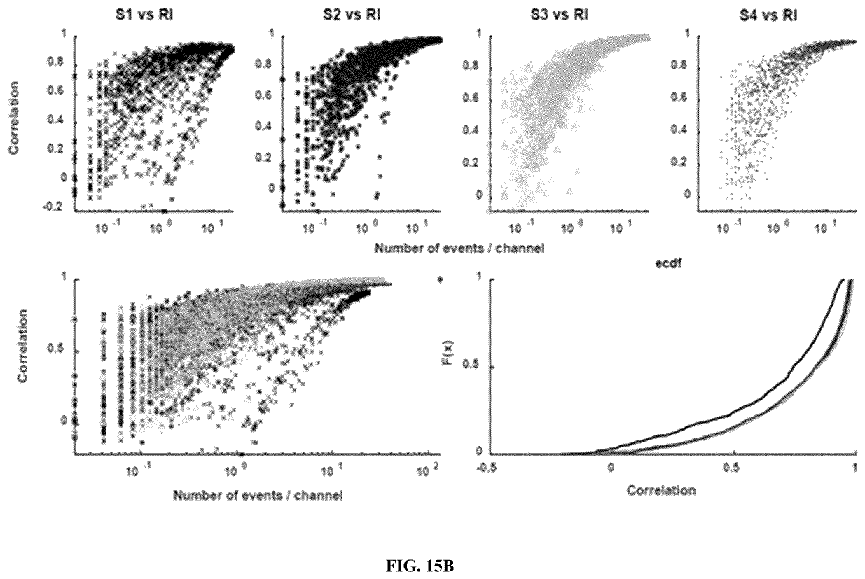

Now, the second factor, which was the number of events per HFO-channel, was used to check whether or not the spatial HFO profiles for S1-4 were fundamentally different. It was noticed that the spatial correlation trends for S1-4 vs. the number of events per HFO-channel monotonically increased until they converged to unity. However, it would be interesting to know how different these trends were for S1-4 before convergence. The empirical cumulative distribution functions (ecdf) of the spatial correlations for S1-4 were estimated and visually inspected. It seemed that five out of seven patients showed ecdfs for S1-4 that were visually not different, as shown in FIG. 15A, while two patients showed trends that were noisy for S1 and visually different for S2-S4 as shown in FIG. 15B.

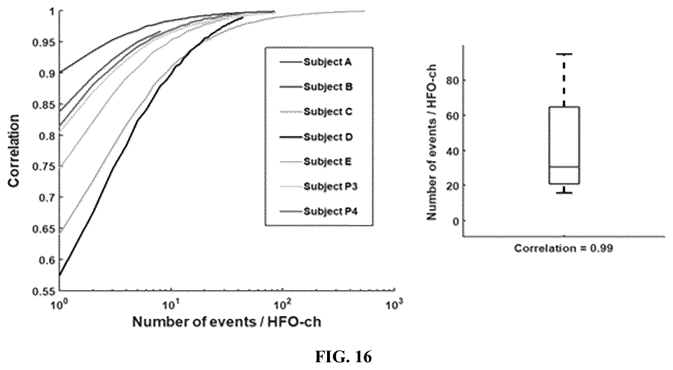

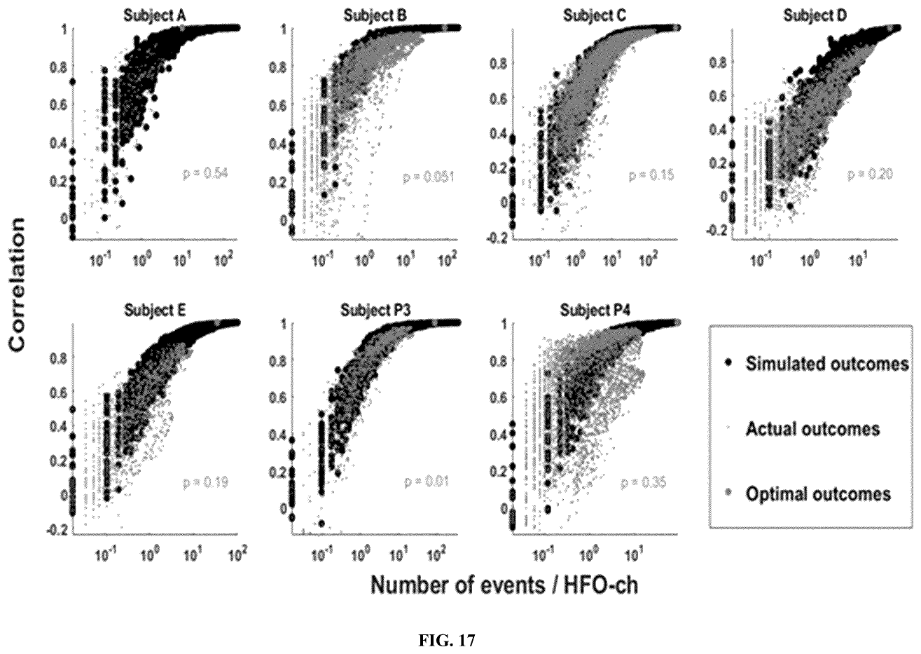

Furthermore, to answer how many HFO samples were adequate, the synthetic data for all patients was initially considered. The mean correlation curves of the synthetic data of seven patients were measured (see FIG. 17, left panel). The minimum numbers of events per channel that gave a correlation of 0.99 or higher was determined. Then the minimum value of that distribution was determined to be fifteen events per channel, and considered as a simulated HFO threshold (see FIG. 16, right panel).

To apply this simulated HFO threshold to the actual data and evaluate its performance, it needs to be established that the synthetic and actual correlation data (pooled S1-S4) are not statistically different. To do that, the synthetic and actual scattered data were binned into ten intervals over the observed range (every 10^{th} percentile from 0-100), and the mean of the data in that interval was computed. The paired t-test was applied between the two data sets formed from the binned data. The test proved that the simulated and actual correlations were mostly not statistically different (p>0.05, n=6 patients) except for patient P3 (see FIG. 17).

In fact, the ratio of candidate HFO channels over the total number of channels of grid and strip electrodes (the proportion of interesting HFO channels) placed on the brain might play an important role in determining the HFO threshold value. Since the actual SOZ area was considered as the main HFO source, then finding the ratio of the SOZ channels over total channels was used to define the range of the proportion of interesting HFO channels indirectly. Table 2 below shows that the ratio of the interesting HFO channels used in this study to determine the HFO threshold was 25%±7.5.

TABLE 2
Information about the number of normal
and seizure channels of each patient.

| | Number of all | Number of | |
|---|---|---|---|
| ID | Channels | SOZ channels | Ratio % |
| A | 48 | 10 | 20 |
| B | 64 | 8 | 13 |
| C | 64 | 14 | 22 |
| D | 55 | 14 | 26 |
| E | 71 | 16 | 23 |
| P3 | 97 | 35 | 36 |

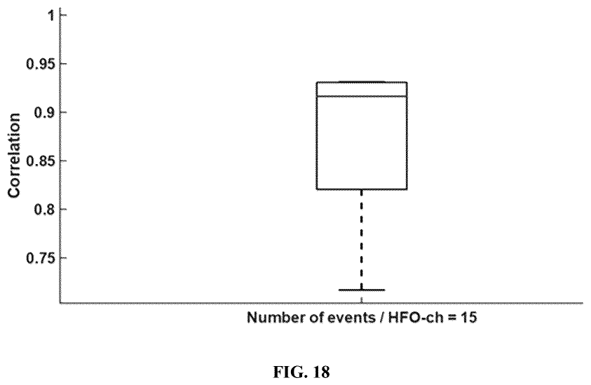

Indeed, when the above simulated HFO threshold (Number of events per HFO-channel=15) was applied to the actual data, the mean of the actual correlation distribution was around 0.9 as shown in FIG. 18.

### Discussion

To understand the effects of vigilance state and sample size on the spatial pattern of HFO activity, a reference image was generated for each subject from fifty percent of the pooled data in S1-4. The spatial correlations between windows having different numbers of epochs and the reference image showed that correlation trends with different numbers of epochs behaved similarly for S1-4, and they all gradually increase and monotonically converge to unity (albeit at different rates). These findings proved that spatial profiles become more consistent and closer to the reference image as the first factor, i.e., the number of epochs, increases regardless of the state. This implies that the number of detected events plays a big role in accurate estimation of the underlying spatial HFO profile. In addition, it was noticed that the mean of the correlation curve would, in most cases, converge more quickly to unity for S4 than for S3 than S2, etc.; in some exceptions they did converge at the same rate, however. This is not unexpected, because even with the same number of epochs, the number of events could decrease progressively from S4 to S1.

Furthermore, when the factor was the number of events per HFO-channel, the correlation monotonically increased until it converged to unity along with less variation regardless of the state. These findings provide further evidence that the spatial profiles become more consistent and closer to the reference image as the number of included events is increased. However, another interesting question arises here: were the spatial profiles fundamentally different for S1-4? Visually, in five of seven patients the ecdf of the correlations for S1-4 had similar trends, while in the other two patients, the ecdf of the correlations had similar trends for S2-4 but not for S1, which was much more scattered. Samples of some detected events in S1 epochs that seemed to spread the correlation were manually checked and it was found that essentially all events were real HFOs, as a consequence of using the 111 detector. Since there were consistent trends in more or less all patients for S2-4 (mainly stages of sleep), samples from sleep may be more consistent and reliable due to the larger average yield, lower likelihood of signal artifacts, and relative certainty with which vigilance state can be determined. However, the more scattered correlations for S1 seen in two patients are most likely due to real physiological HFOs possibly associated with cognitive tasks in wakefulness (e.g., reading, watching TV, etc.); this could be investigated further. These effects suggest that HFO profiles at S1-4 are, in most cases, consistent and not fundamentally different, except in two patients in whom S1 trends were different.

After exploring the question of when to record, it was useful to determine the length of the EEG recording or the number of HFOs that are needed to reliably map the HFO zone. Simulation data was used as a starting point to investigate this problem. All possible numbers of events were created across all HFO-channels, based on the actual spatial HFO distribution of each patient. Synthetic data thus generated for seven patients showed that the correlation versus the number of events per HFO-channel behaves monotonically. For each patient, the number of events per HFO-channel corresponding to the correlation, which was 0.99, was considered as the best-simulated threshold needed to get a good spatial profile to map a reliable HFO zone. Therefore, to set a fixed uniform threshold and use it in the prospective analyses, the lowest value among all thresholds for the seven patients was selected as a threshold, which was 15. In fact, this empirical HFO threshold might be sensitive to the ratio of the number of actual SOZ channels relative to all channels. In this study, this threshold was achieved when the ratio of the SOZ area over the whole area of interest was approximately 25%±7.5%. On the other hand, it was not concentrated or widely separated, for instance, in one channel or across all channels, respectively. It is important to consider this ratio if this simulated threshold is used in future analyses. However, to apply the simulated threshold retrospectively on the existing patients, it was useful to determine how similar the behaviors of the simulated and actual outcomes were. Interestingly, it was found that the simulated and actual outcomes were not significantly different, which means that the simulated threshold could be applied to patient data to examine the actual correlation. The results showed that the range of the actual correlations of seven patients when the simulated threshold was applied was promising (0.78-0.95). These positive outcomes lead us to set the minimum HFO threshold at fifteen and consider using it for future patients to predict a reliable HFO zone.

### Conclusions

Using the HFO detector 111 there is a high likelihood that the detections used in this analysis are genuine HFOs. In general, the results showed that the spatial HFO profiles for states S1-S4 depend on the number of HFO detections across channels. Also, there is no fundamental structural difference in the spatial HFO profile due to vigilance state. However, in a few cases, the profile for S1 appears different from S2-S4. One possible reason for this is that HFOs occurring in S1 might include physiological ripples related to cognitive tasks performed in wakefulness (Arieli, Sterkin et al. 1996, Axmacher, Elger et al. 2008, Hasegawa 2016). In contrast, the HFOs in states S2-S4 are possibly pathological ripples generated by epileptic cortex. In addition, it was concluded that to achieve a reliable and consistent HFO profile for predicting the epileptogenic zone, an adequate number of HFO samples from S2-S4 is needed. However, to save time and effort, S4 was used as a starting point because the probability of seeing HFOs is greatest there, and if the yield happens to be low, then S3 and/or S2 can be included. In terms of HFO samples and the length of recording, the minimum number of HFOs per channel needed to get a reliable HFO profile was estimated at 15 regardless of the length of the recording. Therefore, if this number can be obtained, perhaps adjusted with an appropriate safety factor, from a brief interictal recording at night, there is no need to continue recording for many days to estimate the HFO zone. Overall, these findings could help health care providers map the epileptogenic zone in a short time, with less effort and cost.

All publications, patents, and patent applications mentioned in this specification are herein incorporated by reference to the same extent as if each individual publication, patent, or patent application was specifically and individually indicated to be incorporated by reference, including the references set forth in the following list:

### REFERENCES

1. R. J. Staba, C. L. Wilson, A. Bragin, I. Fried, and J. Engel Jr, “Quantitative analysis of high-frequency oscillations (80-500 Hz) recorded in human epileptic hippocampus and entorhinal cortex,” Journal of neurophysiology, vol. 88, no. 4, pp. 1743-1752, 2002.
2. A. M. McIntosh, R. M. Kalnins, L. A. Mitchell, G. C. Fabinyi, R. S. Briellmann, and S. F. Berkovic, “Temporal lobectomy: long-term seizure outcome, late recurrence and risks for seizure recurrence,” Brain, vol. 127, no. 9, pp. 2018-2030, 2004.
3. J. Wellmer et al., “Risks and benefits of invasive epilepsy surgery workup with implanted subdural and depth electrodes,” Epilepsia, vol. 53, no. 8, pp. 1322-1332, 2012.
4. S. Wiebe, W. T. Blume, J. P. Girvin, and M. Eliasziw, “A randomized, controlled trial of surgery for temporal-lobe epilepsy,” New England Journal of Medicine, vol. 345, no. 5, pp. 311-318, 2001.
5. J. Jacobs et al., “High-frequency oscillations (HFOs) in clinical epilepsy,” Progress in neurobiology, vol. 98, no. 3, pp. 302-315, 2012.
6. P. N. Modur, S. Zhang, and T. W. Vitaz, “Ictal high-frequency oscillations in neocortical epilepsy: implications for seizure localization and surgical resection,” Epilepsia, vol. 52, no. 10, pp. 1792-1801, 2011.
7. M. Zijlmans, P. Jiruska, R. Zelmann, F. S. Leijten, J. G. Jefferys, and J. Gotman, “High-frequency oscillations as a new biomarker in epilepsy,” Ann Neurol, vol. 71, no. 2, pp. 169-78, February 2012.
8. Y. Höller et al., “High-frequency oscillations in epilepsy and surgical outcome. A meta-analysis,” Frontiers in human neuroscience, vol. 9, p. 574, 2015.
9 J. Jacobs, P. LeVan, R. Chander, J. Hall, F. Dubeau, and J. Gotman, “Interictal high-frequency oscillations (80-500 Hz) are an indicator of seizure onset areas independent of spikes in the human epileptic brain,” Epilepsia, vol. 49, no. 11, pp. 18931907, 2008.
10. P. Salami, M. Lévesque, R. Benini, C. Behr, J. Gotman, and M. Avoli, “Dynamics of interictal spikes and high-frequency oscillations during epileptogenesis in temporal lobe epilepsy,” Neurobiology of disease, vol. 67, pp. 97-106, 2014.
11. M. Zijlmans, J. Jacobs, R. Zelmann, F. Dubeau, and J. Gotman, “High-frequency oscillations mirror disease activity in patients with epilepsy,” Neurology, vol. 72, no. 11, pp. 979-986, 2009.
12. C.-G. Bénar, L. Chauvière, F. Bartolomei, and F. Wendling, “Pitfalls of high-pass filtering for detecting epileptic oscillations: a technical note on “false” ripples,” Clinical Neurophysiology, vol. 121, no. 3, pp. 301-310, 2010.
13. C. Haegelen et al., “High-frequency oscillations, extent of surgical resection, and surgical outcome in drug-resistant focal epilepsy,” Epilepsia, vol. 54, no. 5, pp. 848-857, 2013.
14. K. Kerber et al., “Differentiation of specific ripple patterns helps to identify epileptogenic areas for surgical procedures,” Clinical Neurophysiology, vol. 125, no. 7, pp. 1339-1345, 2014.
15 R. Zelmann, M. Zijlmans, J. Jacobs, C.-E. Châtillon, and J. Gotman, “Improving the identification of high frequency oscillations,” Clinical Neurophysiology, vol. 120, no. 8, pp. 1457-1464, 2009.
16 S. V. Gliske, Z. T. Irwin, K. A. Davis, K. Sahaya, C. Chestek, and W. C. Stacey, “Universal automated high frequency oscillation detector for real-time, long term EEG,” Clinical Neurophysiology, vol. 127, no. 2, pp. 1057-1066, 2016.
17. A. F. Al-Bakri, F. Yaghouby, W. Besio, L. Ding, P. Modur, and S. Sunderam, “Effect of Vigilance Changes on the Incidence of High Frequency Oscillations in the Epileptic Brain,” in 2018 40th Annual International Conference of the IEEE Engineering in Medicine and Biology Society (EMBC), 2018, pp. 991-994: IEEE.
18 A. P. Bagshaw, J. Jacobs, P. LeVan, F. Dubeau, and J. Gotman, “Effect of sleep stage on interictal high-frequency oscillations recorded from depth macroelectrodes in patients with focal epilepsy,” Epilepsia, vol. 50, no. 4, pp. 617-628, 2009.
19. G. A. Worrell, K. Jerbi, K. Kobayashi, J.-M. Lina, R. Zelmann, and M. Le Van Quyen, “Recording and analysis techniques for high-frequency oscillations,” Progress in neurobiology, vol. 98, no. 3, pp. 265278, 2012.
20 S. Burnos et al., “Human intracranial high frequency oscillations (HFOs) detected by automatic time-frequency analysis,” PloS one, vol. 9, no. 4, 2014.
21 A. Medvedev, G. Agoureeva, and A. Murro, “A Long Short-Term Memory neural network for the detection of epileptiform spikes and high frequency oscillations,” Scientific Reports, vol. 9, no. 1, pp. 110, 2019.
22 P. Modur and S. Miocinovic, “Interictal high-frequency oscillations (HFOs) as predictors of high frequency and conventional seizure onset zones,” Epileptic Disorders, vol. 17, no. 4, pp. 413-424, 2015.
23. R. Zuo et al., “Automated Detection of High Frequency Oscillations in Epilepsy Based on a Convolutional Neural Network,” Frontiers in computational neuroscience, vol. 13, p. 6, 2019.
24 S. Chaibi, Z. Sakka, T. Lajnef, M. Samet, and A. Kachouri, “Automated detection and classification of high frequency oscillations (HFOs) in human intracereberal EEG,” Biomedical Signal Processing and Control, vol. 8, no. 6, pp. 927-934, 2013.
25 J. Cimbálník, A. Hewitt, G. Worrell, and M. Stead, “The CS algorithm: a novel method for high frequency oscillation detection in EEG,” Journal of neuroscience methods, vol. 293, pp. 6-16, 2018.
26 A. B. Gardner, G. A. Worrell, E. Marsh, D. Dlugos, and B. Litt, “Human and automated detection of high-frequency oscillations in clinical intracranial EEG recordings,” Clinical neurophysiology, vol. 118, no. 5, pp. 1134-1143, 2007.
27 S. Weiss, Z. Waldman, and I. Song, “Signal processing method for distinguishing and characterizing high-frequency oscillations,” ed: Google Patents, 2019.
28 J. A. Blanco et al., “Unsupervised Classification of High-Frequency Oscillations in Human Neocortical Epilepsy and Control Patients,” (in English), Journal of Neurophysiology, vol. 104, no. 5, pp. 2900-2912 November 2010.
29 S. Liu et al., “Exploring the time-frequency content of high frequency oscillations for automated identification of seizure onset zone in epilepsy,” Journal of neural engineering, vol. 13, no. 2, p. 026026, 2016.
30 D. Posada and T. R. Buckley, “Model selection and model averaging in phylogenetics: advantages of Akaike information criterion and Bayesian approaches over likelihood ratio tests,” Systematic biology, vol. 53, no. 5, pp. 793-808, 2004.
31 F. Yaghouby, P. Modur, and S. Sunderam, “Naive scoring of human sleep based on a hidden Markov model of the electroencephalogram,” Conf Proc IEEE Eng Med Biol Soc, vol. 2014, pp. 5028-31, 2014.
32 K. V. Mardia, Statistics of directional data. Academic press, 2014.
33 S. Burnos, B. Frauscher, R. Zelmann, C. Haegelen, J. Sarnthein, and J. Gotman, “The morphology of high frequency oscillations (HFO) does not improve delineating the epileptogenic zone,” Clinical Neurophysiology, vol. 127, no. 4, pp. 2140-2148, 2016.

While the disclosure is susceptible to various modifications and alternative forms, specific embodiments thereof have been shown by way of example in the drawings and are herein described below in detail. It should be understood, however, that the description of specific embodiments is not intended to limit the disclosure to cover all modifications, equivalents and alternatives falling within the spirit and scope of the disclosure as defined by the appended claims.

What is claimed is:

1. A method for identifying high frequency oscillations (HFOs) in neural signals from the brain, the method comprising:
detrending the neural signals; and
identifying HFOs through one or more objective and training-free strike tests selected from the group consisting of (i) amplitude, (ii) rhythmicity, and (iii) ringing.

2. The method of claim 1, wherein the neural signals are obtained using magnetoencephalography (MEG), electroencephalography (EEG), or a combination thereof.

3. The method of claim 1, wherein the neural signals are obtained using electrocorticography (ECoG), intracranial electroencephalography (iEEG), or stereoelectroencephalography (SEEG).

4. The method of claim 1, wherein the identifying step includes the amplitude and ringing strike tests.

5. The method of claim 1, wherein the identifying step includes the amplitude, rhythmicity, and ringing strike tests.

6. A method for monitoring a subject who has been diagnosed as having epilepsy, the method comprising:
identifying HFOs at a first time point and identifying HFOs at a second time point according to the method of claim 1, wherein the neural signals are obtained from the brain of the subject; and
comparing presentation of the HFOs at the first and second time points.

7. The method of claim 6, and further comprising identifying changes in presentation of HFOs.

8. The method of claim 6, wherein the second time point is after receiving treatment for epilepsy.

9. The method of claim 8, wherein the treatment includes one or more of the following: vagus nerve stimulation (VNS), responsive neurostimulation (RNS), deep brain stimulation (DBS), trigeminal nerve stimulation (TNS), anti-seizure medication, sleep medication, medical cannabis, ketogenic diet, and surgery.

10. The method of claim 9, wherein the anti-seizure medication is selected from the group consisting of: Carbamazepine (Tegretol), Valproic Acid (Depakote), Lamotrigine (Lamictal), Levetiracetam (Keppra), Oxcarbazepine (Trileptal), Topiramate (Topamax), Phenobarbital (Luminal), Phenytoin (Dilantin), and Ethosuximide (Zarontin).

11. The method of claim 8, wherein the subject has been identified as having drug-resistant epilepsy (DRE), and the treatment is a neuromodulation or stimulation treatment.

12. The method of claim 11, wherein the treatment includes one or more of the following: vagus nerve stimulation (VNS), responsive neurostimulation (RNS), deep brain stimulation (DBS), and trigeminal nerve stimulation (TNS).

13. The method of claim 8, and further comprising determining treatment efficacy, and maintaining or adjusting treatment, based on changes in presentation of HFOs.

14. A method for predicting whether a subject has epilepsy and/or predicting a location in the brain of the subject that is associated with epilepsy, comprising:
identifying HFOs according to the method of claim 1, wherein the neural signals are obtained from the brain of the subject; and
analyzing presentation of the HFOs.

15. The method of claim 14, wherein presentation of the HFOs includes a presence of HFOs in numbers above a predetermined threshold and/or a presence of HFOs in a determined location.

16. The method of claim 15, and further comprising predicting that the subject has epilepsy and providing treatment to the subject.

17. The method of claim 16, wherein the treatment includes one or more of the following: vagus nerve stimulation (VNS), responsive neurostimulation (RNS), deep brain stimulation (DBS), trigeminal nerve stimulation (TNS), anti-seizure medication, sleep medication, medical cannabis, and ketogenic diet.

18. The method of claim 14, determining a seizure onset zone and/or a specific region involved in generating seizures based upon the analyzed presentation of HFOs.

19. The method of claim 18, and further comprising targeting the onset zone and/or specific region with vagus nerve stimulation (VNS), responsive neurostimulation (RNS), deep brain stimulation (DBS), or trigeminal nerve stimulation (TNS).

20. The method of claim 18, and further comprising determining whether the onset zone and/or specific region is a candidate for surgical intervention.
